SEQUENCE LISTING (30 sequences)

SEQ ID NO: 1 - DNA, 1236 bp, Homo sapiens
atggccctatggtccctgctccatctcaccttcctggggttcagcattaccttgctgttggtccacgggcagggcttccaagggacagcagccatctggccatccctcttcaacgtcaacttgtccaagaaggttcaggaaagcatccagatcccgaacaatgggagtgcgcccctgctcgtggatgtgcgggtgtttgtctccaacgtgtttaatgtggacatcctgcgatacacaatgtcctccatgctgctgcttaggctgtcctggctggacactcgcctggcctggaacactagtgcacacccgcggcacgccatcacgctgccctgggagtctctctggacaccaaggctcaccatcctggaggcgctctgggtggactggagggaccagagcccccaggctcgagtagaccaggacggccacgtgaagctcaacctggccctcgccacggagaccaactgcaactttgagctcctccacttcccccgggaccacagcaactgcagcctcagcttctacgctctcagcaacacggcgatggagttagagttccaggcccacgtggtgaacgagattgtgagtgtcaagagggaatacgtagtttatgatctgaagacccaagtcccaccccagcagctggtgccctgcttccaggtgacgctgaggctgaagaacacggcgctcaagtccatcatcgctctcttggtgcctgcagaggcactgctgttggctgacgtgtgcggggggttgctgcccctccgggccattgagcgcataggctacaaggtgacattgctgctgagttacctcgtcctccactcctccctggtgcaggccctgcccagctcctcctcctgcaacccactgctcatttactacttcaccatcctgctgctgctgctcttcctcagcaccatagagactgtgctgctggctgggctgctggcccggggcaaccttggggccaagagcggccccagcccagccccgagaggggaacagcgagagcacggcaacccagggcctcatcctgctgaagagccctccagaggagtaaaggggtcacagagaagctggcctgagactgctgaccgcatcttcttcctcgtgtatgtggttggggtgctgtgcacccaattcgtctttgcaggaatctggatgtgggcagcgtgcaagtctgacgcagcccctggagaggctgcaccccatggcaggcggcctagactgtaa

SEQ ID NO: 2 - protein, 411 aa, Homo sapiens
MALWSLLHLTFLGFSITLLLVHGQGFQGTAAIWPSLFNVNLSKKVQESIQIPNNGSAPLLVDVRVFVSNVFNVDILRYTMSSMLLLRLSWLDTRLAWNTSAHPRHAITLPWESLWTPRLTILEALWVDWRDQSPQARVDQDGHVKLNLALATETNCNFELLHFPRDHSNCSLSFYALSNTAMELEFQAHVVNEIVSVKREYVVYDLKTQVPPQQLVPCFQVTLRLKNTALKSIIALLVPAEALLLADVCGGLLPLRAIERIGYKVTLLLSYLVLHSSLVQALPSSSSCNPLLIYYFTILLLLLFLSTIETVLLAGLLARGNLGAKSGPSPAPRGEQREHGNPGPHPAEEPSRGVKGSQRSWPETADRIFFLVYVVGVLCTQFVFAGIWMWAACKSDAAPGEAAPHGRRPRL

SEQ ID NO: 3 - DNA, 94 bp, Homo sapiens
atggccctatggtccctgctccatctcaccttcctggggttcagcattaccttgctgttggtccacgggcagggcttccaagggacagcagcca

SEQ ID NO: 4 - protein, 32 aa, Homo sapiens
MALWSLLHLTFLGFSITLLLVHGQGFQGTAAI

SEQ ID NO: 5 - DNA, 125 bp, Homo sapiens
tctggccatccctcttcaacgtcaacttgtccaagaaggttcaggaaagcatccagatcccgaacaatgggagtgcgcccctgctcgtggatgtgcgggtgtttgtctccaacgtgtttaatgtg

SEQ ID NO: 6 - protein, 41 aa, Homo sapiens
WPSLFNVNLSKKVQESIQIPNNGSAPLLVDVRVFVSNVFNV

SEQ ID NO: 7 - DNA, 45 bp, Homo sapiens
gacatcctgcgatacacaatgtcctccatgctgctgcttaggctg

SEQ ID NO: 8 - protein, 15 aa, Homo sapiens
DILRYTMSSMLLLRL

SEQ ID NO: 9 - DNA, 107 bp, Homo sapiens
tcctggctggacactcgcctggcctggaacactagtgcacacccgcggcacgccatcacgctgccctgggagtctctctggacaccaaggctcaccatcctggaggc

SEQ ID NO: 10 - protein, 36 aa, Homo sapiens
SWLDTRLAWNTSAHPRHAITLPWESLWTPRLTILEA

SEQ ID NO: 11 - DNA, 170 bp, Homo sapiens
gctctgggtggactggagggaccagagcccccaggctcgagtagaccaggacggccacgtgaagctcaacctggccctcgccacggagaccaactgcaactttgagctcctccacttcccccgggaccacagcaactgcagcctcagcttctacgctctcagcaacacgg

SEQ ID NO: 12 - protein, 57 aa, Homo sapiens
LWVDWRDQSPQARVDQDGHVKLNLALATETNCNFELLHFPRDHSNCSLSFYALSNTA

SEQ ID NO: 13 - DNA, 125 bp, Homo sapiens
cgatggagttagagttccaggcccacgtggtgaacgagattgtgagtgtcaagagggaatacgtagtttatgatctgaagacccaagtcccaccccagcagctggtgccctgcttccaggtgacg

SEQ ID NO: 14 - protein, 41 aa, Homo sapiens
MELEFQAHVVNEIVSVKREYVVYDLKTQVPPQQLVPCFQVT

SEQ ID NO: 15 - DNA, 211 bp, Homo sapiens
ctgaggctgaagaacacggcgctcaagtccatcatcgctctcttggtgcctgcagaggcactgctgttggctgacgtgtgcggggggttgctgcccctccgggccattgagcgcataggctacaaggtgacattgctgctgagttacctcgtcctccactcctccctggtgcaggccctgcccagctcctcctcctgcaacccactgctca

SEQ ID NO: 16 - protein, 71 aa, Homo sapiens
LRLKNTALKSIIALLVPAEALLLADVCGGLLPLRAIERIGYKVTLLLSYLVLHSSLVQALPSSSSCNPLLI

SEQ ID NO: 17 - DNA, 168 bp, Homo sapiens
tttactacttcaccatcctgctgctgctgctcttcctcagcaccatagagactgtgctgctggctgggctgctggcccggggcaaccttggggccaagagcggccccagcccagccccgagaggggaacagcgagagcacggcaacccagggcctcatcctgctgaag

SEQ ID NO: 18 - protein, 56 aa, Homo sapiens
YYFTILLLLLFLSTIETVLLAGLLARGNLGAKSGPSPAPRGEQREHGNPGPHPAEE

SEQ ID NO: 19 - DNA, 191 bp, Homo sapiens
agccctccagaggagtaaaggggtcacagagaagctggcctgagactgctgaccgcatcttcttcctcgtgtatgtggttggggtgctgtgcacccaattcgtctttgcaggaatctggatgtgggcagcgtgcaagtctgacgcagcccctggagaggctgcaccccatggcaggcggcctagactgtaa

SEQ ID NO: 20 - protein, 62 aa, Homo sapiens
PSRGVKGSQRSWPETADRIFFLVYVVGVLCTQFVFAGIWMWAACKSDAAPGEAAPHGRRPRL

SEQ ID NO: 21 - DNA, 1411 bp, Homo sapiens
atggccctatggtccctgctccatctcaccttcctggggttcagcattaccttgctgttggtccacgggcagggcttccaagggacagcagccatctggccatccctcttcaacgtcaacttgtccaagaaggttcaggaaagcatccagatcccgaacaatgggagtgcgcccctgctcgtggatgtgcgggtgtttgtctccaacgtgtttaatgtggacatcctgcgatacacaatgtcctccatgctgctgcttaggctgtcctggctggacactcgcctggcctggaacactagtgcacacccgcggcacgccatcacgctgccctgggagtctctctggacaccaaggctcaccatcctggaggcgctctgggtggactggagggaccagagcccccaggctcgagtagaccaggacggccacgtgaagctcaacctggccctcgccacggagaccaactgcaactttgagctcctccacttcccccgggaccacagcaactgcagcctcagcttctacgctctcagcaacacggcgatggagttagagttccaggcccacgtggtgaacgagattgtgagtgtcaagagggaatacgtagtttatgatctgaagacccaagtcccaccccagcagctggtgccctgcttccaggtgacgctgaggctgaagaacacggcggaattccggcggcccctcttctatgtggtcagcctgctactgcccagcatcttcctcatggtcatggacatcgtgggcttctacctgccccccaacagtggcgagagggtctctttcaagattacactcctcctgggctactcggtcttcctgatcatcgtttctgacacgctgccggccactgccatcggcactcctctcattggtgtctactttgtggtgtgcatggctctgctggtgataagtttggccgagaccatcttcattgtgcggctggtgcacaagcaagacctgcagcagcccgtgcctgcttggctgcgtcacctggttctggagagaatcgcctggctactttgcctgagggagcagtcaacttcccagaggcccccagccacctcccaagccaccaagactgatgactgctcagccatgggaaaccactgcagccacatgggaggaccccaggacttcgagaagagcccgagggacagatgtagccctcccccaccacctcgggaggcctcgctggcggtgtgtgggctgctgcaggagctgtcctccatccggcaattcctggaaaagcgggatgagatccgagaggtggcccgagactggctgcgcgtgggctccgtgctggacaagctgctattccacatttacctgctggcggtgctggcctacagcatcaccctggttatgctctggtccatctggcagtacgctc

SEQ ID NO: 22 - DNA, 1269 bp, Homo sapiens
atgctgctgtgggtccagcaggcgctgctcgccttgctcctccccacactcctggcacagggagaagccaggaggagccgaaacaccaccaggcccgctctgctgaggctgtcggattaccttttgaccaactacaggaagggtgtgcgccccgtgagggactggaggaagccaaccaccgtatccattgacgtcattgtctatgccatcctcaacgtggatgagaagaatcaggtgctgaccacctacatctggtaccggcagtactggactgatgagtttctccagtggaaccctgaggactttgacaacatcaccaagttgtccatccccacggacagcatctgggtcccggacattctcatcaatgagttcgtggatgtggggaagtctccaaatatcccgtacgtgtatattcggcatcaaggcgaagttcagaactacaagccccttcaggtggtgactgcctgtagcctcgacatctacaacttccccttcgatgtccagaactgctcgctgaccttcaccagttggctgcacaccatccaggacatcaacatctctttgtggcgcttgccagaaaaggtgaaatccgacaggagtgtcttcatgaaccagggagagtgggagttgctgggggtgctgccctactttcgggagttcagcatggaaagcagtaactactatgcagaaatgaagttctatgtggtcatccgcgaattcctcaagtccatcatcgctctcttggtgcctgcagaggcactgctgttggctgacgtgtgcggggggttgctgcccctccgggccattgagcgcataggctacaaggtgacattgctgctgagttacctcgtcctccactcctccctggtgcaggccctgcccagctcctcctcctgcaacccactgctcatttactacttcaccatcctgctgctgctgctcttcctcagcaccatagagactgtgctgctggctgggctgctggcccggggcaaccttggggccaagagcggccccagcccagccccgagaggggaacagcgagagcacggcaacccagggcctcatcctgctgaagagccctccagaggagtaaaggggtcacagagaagctggcctgagactgctgaccgcatcttcttcctcgtgtatgtggttggggtgctgtgcacccaattcgtctttgcaggaatctggatgtgggcagcgtgcaagtctgacgcagcccctggagaggctgcaccccatggcaggcggcctagactg

SEQ ID NO: 23 - protein, 470 aa, Homo sapiens [sequence omitted]

SEQ ID NO: 24 - protein, 423 aa, Homo sapiens
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIREFLKSIIALLVPAEALLLADVCGGLLPLRAIERIGYKVTLLLSYLVLHSSLVQALPSSSSCNPLLIYYFTILLLLLFLSTIETVLLAGLLARGNLGAKSGPSPAPRGEQREHGNPGPHPAEEPSRGVKGSQRSWPETADRIFFLVYVVGVLCTQFVFAGIWMWAACKSDAAPGEAAPHGRRPRL

SEQ ID NO: 25 - DNA, 22 bp, Artificial Sequence
  oligonucleotide primer
caactgcagcctcagcttctac

SEQ ID NO: 26 - DNA, 29 bp, Artificial Sequence
  oligonucleotide probe
ctcagcaacacggcgatggagttagagtt

SEQ ID NO: 27 - DNA, 24 bp, Artificial Sequence
  oligonucleotide primer
tcttgacactcacaatctcgttca

SEQ ID NO: 28 - DNA, 22 bp, Artificial Sequence
  oligonucleotide primer
caactgcagcctcagcttctac

SEQ ID NO: 29 - DNA, 29 bp, Artificial Sequence
  oligonucleotide probe
ctcagcaacacggcgatggagttagagtt

SEQ ID NO: 30 - DNA, 24 bp, Artificial Sequence
  oligonucleotide primer
tcttgacactcacaatctcgttca

(19) United States
(12) Patent Application Publication
(10) Pub. No.: US 2006/0166310 A1
(43) Pub. Date: Jul. 27, 2006

(54) Serotonin receptor

(75) Inventors: Anna E Lobley, London (GB); David Michalovich, London (GB); Ilana Stancovski, London (GB); Kathryn E Allen, London (GB); Janet M Allen, London (GB); Vadym N Osypenko, Glasgow (GB); Alison M Gurney, Glasgow (GB)

Correspondence Address: DARBY & DARBY P.C., P. O. BOX 5257, NEW YORK, NY, 10150-5257, US

(73) Assignee: Inpharmatica Ltd., London (GB)

(21) Appl. No.: 10/520,052

(22) PCT Filed: Jul. 21, 2003
(86) PCT No.: PCT/GB03/03130

§ 371 Date: Sep. 26, 2005

(30) Foreign Application Priority Data
Jul. 19, 2002 (GB) 0216903.5

(51) Int. Cl. C07K 14/72; C07H 21/04; C12P 21/06; C07K 14/705; C07K 16/28

(52) U.S. Cl. 435/69.1; 435/320.1; 435/325; 530/350; 530/388.22; 536/23.5

(57) ABSTRACT

This invention relates to a protein, termed INPIONCH1, herein identified as a 5-HT3 receptor and to the use of this protein and nucleic acid sequence from the encoding gene in the diagnosis, prevention and treatment of disease.

This invention relates to a novel protein, termed INPIONCH1, herein identified as a member of the 5-HT3 (serotonin) receptor group, and to the use of this protein and nucleic acid sequence from the encoding gene in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Introduction to Ligand Gated Ion Channels

Ligand gated ion channels (LGICs), or neurotransmitter gated ion channels are integral membrane proteins of the ion channel superfamily (Derkach et al Trends in Pharmacological Science (1992) 13(10):391-397). Channels are characterised by their structure, by their ion selectivity and by their gating mechanism. The LGIC family is a highly homologous gene family, which can be subdivided into anion channels (glutamate, GABA and glycine gated chloride channels), and cation selective channels (5-HT3 and acetylcholine receptors). 5-HT3 receptors are pentameric assemblies that are selective for cations such as Na, K and Ca ions, and are gated by the extracellular neurotransmitter, mitogen and hormone, serotonin. 5-HT3 receptors are found predominantly in the brain in the hippocampus, amygdala and superficial layers of the cerebral cortex. Lower transcript levels have also been reported in colon (Lankiewicz et al Molecular Pharmacology 1998 53: 202-212) kidney, spleen, tonsil, uterus, prostate, ovary, placenta and intestine (see Jackson and Yakel in Annual Review of Physiology 1995, 57: 447-468; Fletcher and Barnes in Trends in Pharmacological Science 1998, 19: 212-215).

Each receptor subunit shares similar topology and organisation around the membrane. The first 20-25 amino acids contain a signal peptide followed by the extracellular ligand-binding domain. The ligand binding domain contains a Cys-loop motif conserved throughout the ligand gated ion channel family and a conserved Tryptophan thought to be important in binding curare (Yan et al Journal of Biological Chemistry 1999, 9: 5537-5541). Four transmembrane helices (M1-M4) are predicted to form the transmembrane domain in the second half of the molecule with a large intracellular loop region between M3 and M4. The channel pore forming region is located in the M2 helix which is thought to be hinged by a conserved Leu 251 (Barnes et al Neuropharmacology 1999, 38: 1083-1152). Each subunit is made up of 9 exons conserved in terms of number and length throughout the 5-HT3 family (Uetz et al FEBS Letters 1994, 339: 302-306; Bruss et al Neuropharmacology 2000, 39: 308-315).

Cytogenetic locations for the 5-HT3A and 5-HT3B receptor subunits have been established (Weiss et al Genomics 1995, 29: 304-305 and Davies et al Nature 1999, 397: 359-363) on chromosome 11q23. Evidence of functional heteromultimeric channels (Fletcher and Barnes, British Journal of Pharmacology 1997 122: 655-662), together with the existence of splice variants, short and long isoforms of the 5-HT3A subunit, (Bruss et al Annals of the New York Academy of Science 1998, 861: 234-235) accounts for some of the diversity observed in channel properties, such as activation speed, ion selectivity, and conductance. Receptor purification by affinity chromatography revealed at least two protein bands with molecular masses 54 (5-HT3A) and 38 kDa respectively (Belelli et al Mol Pharmacol. 1995 48(6):1054-62), and affinity purified 5-HT3 receptor, solubilized from pig cerebral cortex, showed at least three separate components using silver staining of protein on denaturing gels (Fletcher and Barnes, British Journal of Pharmacology 1997, 122: 655-662). This evidence suggests that 5-HT3 receptors may be composed of more than two types of subunit.

The serotonergic pathway is heavily implicated in the processes of learning and memory, food intake, sexual behaviour and emotions, (Buhot et al Ann Med. 2000, 32(3): 210-212) and therefore 5-HT3 receptors are molecular drug targets in the treatment of a variety of disorders. These include obesity, anorexia, autism, alcoholism (Johnson et al Alcohol Clin Exp Res 2000, 24(5): 737-742), and psychiatric disorders such as depression, schizophrenia, panic attacks, and behavioural consequences of drug abuse (Greenshaw and Silverstone, Drugs 1997, 53(1): 20-39). 5-HT3 receptor agonists and antagonists are also targets for antiemitic drugs (granisetron, ondansetron) that treat nausea induced by anticancer therapies, general anesthesia, (Gandara et al, Support Care Cancer 1998, 6(3):237-243), anxiety related disorders, and vertigo. These agonists and antagonists are also used to treat pain in migraine (Ferrari M D J Neurology 1991, 238: Suppl 1:S53-6), rheumatoid arthritis (Stratz Scand J Rheumatology Suppl. 2000, 113: 66-71) irritable bowel syndrome (Talley N J Am J Manag Care 2001 8 Suppl S261-7) and constipation. Many of the compounds which modulate the function of the 5-HT3 receptor, such as alcohol, (Narahashi et al Alcohol Clin Exp Res. 2001 25(5 Suppl ISBRA):182S-188S), steroids (Zinder et al Acta Physiol Scand. 1999, 167(3):181-8) and anaesthetic barbiturates (Miller et al Eur J Anaesthesiol. 1995, 12(1): 21-30), are common to other members of the ligand gated ion channel superfamily, suggesting functional as well as sequence based similarities between receptors. Indeed the acetylcholine receptors, the most closely related receptors to the 5-HT3 channels are involved in disease pathways such as Alzheimer's Disease (Rosecrans et al Res Monogr. 1991, 116:101-16), Parkinson's Disease and Huntington's Disease (for review see Lindstrom Molecular Neurobiology 1997, 15(2):193-222).

Alteration of the activity of members of the 5-HT3 receptor group, in particular of 5-HT3 receptor subunits, thus provides a means to alter the disease phenotype. Identification of new members of the 5-HT3 receptor group and of 5-HT3 receptor subunits is therefore of extreme importance in increasing understanding of the underlying mechanisms of pain reception, sickness, psychosis and addiction, and in developing more effective gene or drug therapies to treat these disorders.

### THE INVENTION

The invention is based on the discovery that the INPIONCH1 protein functions as a member of the 5-HT3 receptor group, preferably as a 5-HT3 receptor subunit. The INPIONCH1 protein is a 421 amino acid protein encoded by 9 exons located on human chromosome 17q25.1.

The INPIONCH1 protein is a non-selective cation channel predicted to contain four transmembrane spanning regions and a signal peptide in the first 25 amino acids, specifically amino acids 1-23. Sequence alignment of INPIONCH1 with other ion channels shows that it is most closely related to a 5-HT3-b receptor subunit from the mouse. The length and content of the INPIONCH1 exons are similar to the conserved exon structure found in human and murine 5-HT3 subunits. The INPIONCH1 protein has been cloned and expressed and patch-clamping studies presented herein confirm that it functions as a serotonin-gated non-specific cation channel. In view of the fact that serotonin-gated (5-HT3) ion channels have been implicated in a wide-range of diseases,

the INPIONCH1 protein is an important target for further biochemical characterisation.

As described in more detail in the examples, the cloning of INPIONCH1 proved problematic using standard methods due to the rarity of the INPIONCH1 transcript and the fact that primers designed using standard techniques resulted in the amplification of transcripts unrelated to INPIONCH1. The comprehensive disclosure in the examples of how to successfully clone INPIONCH1 enables the skilled person to conduct high-level expression, purification and characterisation of the INPIONCH1 polypeptides of the invention for further study.

Results presented herein clearly indicate that the INPIONCH1 transcript is present at detectable levels in only a few human tissues and cell lines, being present in the brain, thymus and Jurkat cells. The identification of the INPIONCH1 transcript in foetal and adult thymus is consistent with a role for this channel in regulating T lymphocyte function or maturation, particularly in view of the fact that expression levels appeared to be higher in foetal thymus compared to adult. It has previously been shown that other 5-HT3 agonists can modulate T cell activation through triggering influx of sodium (Khan et al, 1999, J. Biochem., 344 199-204); although the molecular target mediating this useful effect was not identified in this study. The INPIONCH1 protein may be responsible for this pharmacology and its identification and cloning will enable the skilled person to further define mechanisms underlying T cell activation and thus develop therapeutic applications.

The particular tissues and cell lines identified herein as expressing the INPIONCH1 protein represent ideal targets for further studies of the mechanism of action of INPIONCH1 in vivo. Such studies may, for example, make use of ligands identified using the assays and screening methods disclosed herein to investigate the effects of inducing or inhibiting INPIONCH1 protein function.

Agonists and antagonists of INPIONCH1 are likely to be of great value in the treatment of diseases in which members of the 5-HT3 receptor group are implicated. Agonists and antagonists of the INPIONCH1 protein can be readily identified using the assays and screening methods disclosed herein. Once identified, the effect of agonists and antagonists on diseased cell lines and tissue types may then be investigated using the methods disclosed herein or known to those of skill in the art. It is likely that certain agonists or antagonists identified using the assays and methods disclosed herein will be useful in the prophylaxis or treatment of diseases associated with the INPIONCH1 protein.

The finding that INPIONCH1 is expressed in the thymus and I cells suggests that compounds that interfere with ion pore function or act as ligand binding domain agonists or antagonists will have value in the treatment of diseases associated T cells such as inflammatory bowel diseases (including Crohns disease and ulcerative colitis), multiple sclerosis, psoriasis, rheumatoid arthritis, thrombocytopenia, type I diabetes mellitus, asthma, atopic dermatitis, atopic rhinitis and conjunctivitis. Ligand binding domain agonists or antagonists or compounds that interfere with pore function may also be able to promote T cell activation and thus be used to treat diseases in which regulation of T cell activation is required, such as cancers, viral infections, bacterial infections (including tuberculosis) and fungal infections. In addition, ligand binding domain agonists or antagonists or compounds that interfere with ion pore function may be of value in the treatment of diseases associated with T cell proliferation such as leukaemia and diseases associated with T cell depletion such as HIV infection, chemotherapy and radiotherapy.

The suggestion that agonists and antagonists of the INPIONCH1 protein may be useful in the treatment of these diseases is supported by results presented herein that transcript levels for the INPIONCH1 protein are higher than normal in tissue from patients with inflammatory bowel disease, and lower than normal in tissue from patients with psoriasis and rheumatoid arthritis. It is possible that antagonists of INPIONCH1 protein may be useful in the prophylaxis or treatment of inflammatory bowel disease and that agonists of the INPIONCH1 protein may be useful in the prophylaxis and treatment of psoriasis and rheumatoid arthritis. The suggestion that the INPIONCH1 protein may be involved in the pathology of psoriasis is supported by the fact that a psoriasis susceptibility locus named PSOR2 (OMIM:602723) is known to exists on chromosome 17q25, the chromosomal location to which the INPIONCH1 has been mapped.

The finding that levels of INPIONCH1 expression are higher in tissue from patients with inflammatory bowel disease and lower in patients with psoriasis and rheumatoid arthritis also suggests that it may be possible to develop diagnostic tests for these diseases based on the detection of the INPIONCH1 protein or the transcript encoding it.

In a first aspect, the invention provides a polypeptide sequence, which polypeptide:
(i) comprises the amino acid sequence as recited in SEQ ID NO:2;
(ii) is a fragment thereof having 5-HT3 protein function, or having an antigenic determinant in common with the polypeptides of (i); or
(iii) is a functional equivalent of (i) or (ii).

The polypeptide having the sequence recited in SEQ ID NO:2 is referred to hereafter as “the INPIONCH1 polypeptide”. Preferably, a polypeptide according to the invention consists of the amino acid sequence as recited in SEQ ID NO:2, or is a variant thereof.

A preferred fragment according to part ii) above includes the INPIONCH1 polypeptide without its predicted signal sequence, extending between amino acids 24 to 421 of SEQ ID NO:2. Further preferred fragments according to part ii) above include the ligand binding domain of the INPIONCH1 polypeptide extending from residues 24-229 of SEQ ID NO:2 and the pore forming region, also referred to herein as the transmembrane domain, of INPIONCH1 extending from residues 230 to 421 of SEQ ID NO:2. These fragments may be used in the screening assays described below to identify ligands of the INPIONCH1 and compounds which act as agonists or antagonists of the INPIONCH1 protein function.

This aspect of the invention also includes fusion proteins that incorporate polypeptide fragments and functional equivalents of these fragments, provided that said fusion proteins possess activity as a ligand-gated ion channel, preferably the activity of a member of the 5-HT3 receptor group. A preferred fusion protein is a receptor chimera comprising the ligand binding domain of the INPIONCH1 polypeptide and the transmembrane domain of a different ligand-gated ion channel, preferably of another member of the 5-HT3 receptor group, such as a human 5-HT3a or human 5-HT3b subunit. Alternatively, a receptor chimera may comprise the transmembrane domain of the INPIONCH1 polypeptide and the ligand binding domain of a different 5-HT3 receptor, such as a human 5-HT3a or human 5-HT3b subunit. An exemplary receptor chimera comprising the ligand binding domain of the INPIONCH1 polypeptide and the transmembrane domain of a human 5-HT3a subunit comprises the amino acid sequence as recited in SEQ ID NO:23. An exemplary receptor chimera comprising the ligand binding domain of a human 5-HT3a subunit and the transmembrane domain of the INPIONCH1 polypeptide comprises the amino acid sequence as recited in SEQ ID NO:24.

Such receptor chimeras are useful to study the function of different domains of the INPIONCH1 polypeptide.

When referring to polypeptides having “5-HT3 protein function”, we refer to polypeptides that are members of the 5-HT3 receptor group and have “5-HT3 receptor function” or “5-HT3 receptor activity”. These polypeptides comprise amino acid sequence or structural features that can be identified as conserved features present in both INPIONCH1 and other known members of the 5-HT3 receptor group, and that also retain their ligand specificity such that ligand-gated ion movement is not substantially reduced in comparison to the naturally-occurring INPIONCH1. Such activity can be determined using methods that are standard in the art which are described herein. For example, the ability of a polypeptide to bind serotonin may be assessed by expressing the polypeptide on the surface of a cell or anchoring it to a polypeptide, incubating it with labelled serotonin and detecting the binding of serotonin to the polypeptide by detecting the label. The ability of a polypeptide to promote ion movement may be assessed by expressing the polypeptide in cells preloaded with an ion-sensitive dye (such as sodium-sensitive SBFI, potassium sensitive PBFI, or calcium sensitive Fura2 or Fluo3) and determining the spectral response of these cells on addition of serotonin. Preferably, binding of serotonin to the polypeptides of the invention induces an influx of sodium ions.

Preferably, the INPIONCH1 polypeptide or functional equivalent thereof is a 5-HT3 receptor subunit. According to this embodiment of this first aspect of the invention, the INPIONCH1 polypeptide or functional equivalent may form a homopentamer or a heteropentamer. Heteropentamers may be formed by the multimerisation of a 5-HT3 receptor subunit which is a INPIONCH1 polypeptide or functional equivalent thereof with subunits from other ligand-gated ion channels, including subunits from other 5-HT3 receptors.

In a second aspect, the invention provides a purified nucleic acid molecule which encodes a polypeptide of the first aspect of the invention. Preferably, the purified nucleic acid molecule has the nucleic acid sequence as recited in SEQ ID NO: 1 (encoding the INPIONCH1 polypeptide). Where the polypeptide is a fusion protein encoding a receptor chimera, the purified nucleic acid molecule preferable has the nucleic acid sequence recited in SEQ ID NO:21 (coding for SEQ ID NO:23) or the nucleic acid sequence recited in SEQ ID NO:22 (coding for SEQ ID NO:24).

In a third aspect, the invention provides a purified nucleic acid molecule which hybridises under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention. In a sixth aspect, the invention provides a ligand which binds specifically to, and which preferably modulates the 5-HT3 receptor activity of, a polypeptide of the first aspect of the invention. Ligands may either activate or inhibit (antagonise) or activate (agonise) the activity of a polypeptide of a first aspect of the invention.

Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

Preferred ligands bind specifically to, and selectively modulate the activity of a INPIONCH1 polypeptide of the invention either by binding to the ligand binding domain or to the pore forming region. Examples of ligands include desensitisers, blockers and allosteric modulators.

Specific ligands may be identified by performing a binding assay in which the INPIONCH1 polypeptide is anchored on the cell surface or a solid matrix support (beads, plates, etc) and is incubated with a labelled or unlabeled candidate ligand. The binding of the ligand may be detected either directly by an increase in the amount of label (for example fluorescence), or any other means (for example a signal transmitted inside the cells). Alternative methods of identifying ligands are described herein.

In a seventh aspect, the invention provides a compound that is effective to alter the expression of a natural gene which encodes a polypeptide of the first aspect of the invention or to regulate the activity of a polypeptide of the first aspect of the invention.

In a seventh aspect, the invention provides a compound that is effective to alter the expression of a natural gene which encodes a polypeptide of the first aspect of the invention or to regulate the activity of a polypeptide of the first aspect of the invention.

Such compounds may be identified using the assays and screening methods disclosed herein.

A compound of the seventh aspect of the invention may either increase (agonise) or decrease (antagonise) the level of expression of the gene or the activity of the polypeptide. Importantly, the identification of the function of the INPIONCH1 polypeptide allows for the design of screening methods capable of identifying compounds that are effective in the treatment and/or diagnosis of certain diseases, in particular diseases in which members of the 5-HT3 receptor group are implicated and diseases associated with T cells, including diseases associated with T cell proliferation or depletion or diseases which treated by regulating T cell activation. Examples of suitable assays and screening methods are provided herein.

In an eighth aspect, the invention provides a polypeptide of the first aspect of the invention, or a nucleic acid molecule of the second or third aspect of the invention, or a vector of the fourth aspect of the invention, or a host cell according to the fifth aspect of the invention, or a ligand of the sixth aspect of the invention, or a compound of the seventh aspect of the invention, for use in therapy or diagnosis. These molecules may also be used in the manufacture of a medicament for the treatment of certain diseases, preferably disease in which members of the 5-HT3 receptor family are implicated, including, but not limited to nausea, vomiting, pain, eating disorders, alcoholism, psychosis, side effects of various anticancer therapies, irritable bowel syndrome, gastrointestinal related disorders, Alzheimer's disease, Parkinson's disease, Huntington's disease, cognitive disorders, behavioural disorders and phobias such as anxiety related illnesses and addiction, obsessive compulsive behaviour, memory and learning disorders, depression and panic disorders, depression and panic disorders, asthma, inflammation, sexual dysfunction, disorders of the neuroendocrine and cardiovascular systems.

These molecules may also be useful in the treatment of diseases associated T cells such as inflammatory bowel diseases (including Crohns disease and ulcerative colitis), multiple sclerosis, psoriasis, rheumatoid, thrombocytopenia, type I diabetes mellitus, asthma, atopic dermatitis, atopic rhinitis and conjunctivitis, diseases associated with T cell proliferation such as leukaemias, diseases associated with T-cell depletion such as HIV infection, chemotherapy and radiotherapy, and diseases where regulation of T cell activation is required, such as cancers, viral infections, bacterial infections (including tuberculosis) and fungal infections.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

Preferably, the disease diagnosed by a method of the ninth aspect of the invention is a disease in which members of the 5-HT3 receptor group are implicated, as described above. The disease may be a disease associated T cells such as inflammatory bowel diseases (including Crohns disease and ulcerative colitis), multiple sclerosis, psoriasis, rheumatoid, thrombocytopenia, type I diabetes mellitus, asthma, atopic dermatitis, atopic rhinitis and conjunctivitis, a disease associated with T cell proliferation such as leukaemia, a disease associated with T-cell depletion such as HIV infection, chemotherapy and radiotherapy, and a disease where regulation of T cell activation is required, such as cancers, viral infections, bacterial infections (including tuberculosis) and fungal infections. Where the disease is psoriasis or rheumatoid arthritis, the method of diagnosis may involving detecting a level of expression that is lower than the control level, whereas, when the disease is inflammatory bowel disease, the method of diagnosis may involving detecting a level of expression that is higher than the control level.

In a tenth aspect, the invention provides for the use of a polypeptide of the first aspect of the invention as a member of the 5-HT3 receptor group, preferably as a 5-HT3 subunit molecule. Suitable uses of a polypeptide of the invention as a member of the 5HT-3 receptor group include the promotion of serotonin-mediated cation movement into and out of cells. Other suitable uses of the polypeptides of the invention include use in methods for the identification of ligands which bind the ligand binding domain or the pore forming region of INPIONCH1, which ligands will be of use in the modulation of disease processes in which INPIONCH1 is implicated. INPIONCH1 may be involved in T cell maturation and the polypeptides of the invention may also be used as a research tool into T cell maturation.

The invention also provides for the use of a nucleic acid molecule according to the second or third aspects of the invention to express a protein that possesses activity as a member of the 5-HT3 receptor group. Such nucleic acid molecules are of utility in the production of the polypeptides of the invention, which polypeptides are useful in a variety of situations, as described above.

The invention also provides a method for effecting the activity of a member of the 5-HT3 receptor group, said method utilising a polypeptide of the first aspect of the invention, or a fragment thereof.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell according to the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell according to the fifth aspect of the invention, or a ligand of the sixth aspect of the invention, or a compound of the seventh aspect of the invention, for use in the manufacture of a medicament for the diagnosis or treatment of a disease, preferably a disease in which 5-HT3 receptors are implicated, including nausea, vomiting, pain, eating disorders, alcoholism, psychosis, side effects of various anticancer therapies, irritable bowel syndrome, gastrointestinal related disorders, Alzheimer's disease, Parkinson's disease, Huntington's disease, cognitive disorders, behavioural disorders and phobias such as anxiety related illnesses and addiction, obsessive compulsive behaviour, memory and learning disorders, depression and panic disorders, asthma, inflammation, sexual dysfunction, disorders of the neuroendocrine and cardiovascular systems.

The invention also provides a polypeptide of the first aspect of the invention, or a nucleic acid molecule of the second or third aspect of the invention, or a vector of the fourth aspect of the invention, or a host cell according to the fifth aspect of the invention, or a ligand of the sixth aspect of the invention, or a compound of the seventh aspect of the invention, for use in the manufacture of a medicament for the diagnosis or treatment of diseases associated T cells such as inflammatory bowel diseases (including Crohns disease and ulcerative colitis), multiple sclerosis, psoriasis, rheumatoid arthritis, thrombocytopenia, type I diabetes mellitus, asthma, atopic dermatitis, atopic rhinitis and conjunctivitis, diseases associated with T cell proliferation such as leukaemias, diseases associated with T-cell depletion such as HIV infection, chemotherapy and radiotherapy, and diseases where regulation of T cell activation is required, such as cancers, viral infections, bacterial infections (including tuberculosis) and fungal infections.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell according to the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

Preferably, the disease is a disease in which members of the 5-HT3 receptor group are implicated or a disease associated with T cell function, T cell proliferation, T cell depletion, or a disease in which regulation of T cell activation would be desirable, as described above. Results presented herein suggest that expression of the INPIONCH1 polypeptide is higher in patients with inflammatory bowel disease compared to healthy patients and that antagonists of the polypeptide of the first aspect of the invention may be useful in the treatment of inflammatory bowel disease. Conversely, results presented herein suggest that expression of the INPIONCH1 polypeptide is lower in patients with psoriasis or rheumatoid arthritis compared to healthy patients and that agonists of the polypeptides of the first aspect of the invention may be useful in the treatment of these diseases.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be using in screening regimes for the identification of compounds that are effective in the treatment or diagnosis of such a disease. The INPIONCH1 gene is only present as a pseudogene in mice and rats such that it is unnecessary to knock-out a native gene before introducing an INPIONCH1 gene from another species into a mouse or rat.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro- protein that can be activated by cleavage of the pre-, pro- or prepro- portion to produce an active mature polypeptide. In such polypeptides, the pre-, pro- or prepro- sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INPIONCH1 polypeptide. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INPIONCH1 polypeptide. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues includes a substituent group.

Typically, greater than 30% identity between two polypeptides is considered to be an indication of functional equivalence. Preferably, functionally equivalent polypeptides of the first aspect of the invention have a degree of sequence identity with the INPIONCH1 polypeptide, or with active fragments thereof, of greater than 30%. More preferred polypeptides have degrees of identity of greater than 40%, 50%, 60%, 70%, 80%, 90%, 95%, 98% or 99%, respectively.

Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

The functionally-equivalent polypeptides of the first aspect of the invention may also be polypeptides which have been identified using one or more techniques of structural alignment. For example, the Inpharmatica Genome Threader™ technology that forms one aspect of the search tools used to generate the Biopendium search database may be used (see PCT application published as WO 01/67507) to identify polypeptides of presently-unknown function which, while having low sequence identity as compared to the INPIONCH1 polypeptide, are predicted to have 5-HT3 receptor activity, by virtue of sharing significant structural homology with the INPIONCH1 polypeptide sequence.

By “significant structural homology” is meant that the Inpharmatica Genome Threader™ predicts two proteins, or protein regions, to share structural homology with a certainty of at least 10% more preferably, at least 20%, 30%, 40%, 50%, 60%, 70%, 80%, 90% and above. The certainty value of the Inpharmatica Genome Threader™ is calculated as follows. A set of comparisons was initially performed using the Inpharmatica Genome Threader™ exclusively using sequences of known structure. Some of the comparisons were between proteins that were known to be related (on the basis of structure). A neural network was then trained on the basis that it needed to best distinguish between the known relationships and known not-relationships taken from the CATH structure classification (www.biochem.ucl.ac.uklbsm/cath). This resulted in a neural network score between 0 and 1. However, again as the number of proteins that are related and the number that are unrelated were known, it was possible to partition the neural network results into packets and calculate empirically the percentage of the results that were correct. In this manner, any genuine prediction in the Biopendium search database has an attached neural network score and the percentage confidence is a reflection of how successful the Inpharmatica Genome Threader™ was in the training/testing set.

The polypeptides of the first aspect of the invention also include fragments of the INPIONCH1 polypeptide and fragments of the functional equivalents of the INPIONCH1 polypeptide, provided that those fragments retain ligand gated ion channel receptor activity, specifically 5-HT3 receptor activity, or have an antigenic determinant in common with the INPIONCH1 polypeptide.

As used herein, the term “fragment” refers to a polypeptide having an amino acid sequence that is the same as part, but not all, of the amino acid sequence of the INPIONCH1 polypeptide or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known members of the 5-HT3 receptor group.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known members of the 5-HT3 receptor group.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography. Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode the polypeptide sequences recited in SEQ ID NO:2, and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes the polypeptide of SEQ ID NO:2 may be identical to the coding sequence of the nucleic acid molecule shown in SEQ ID NO:1, may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Examples of suitable fragments of nucleic acid molecules according to the invention include those encoding the separate exons for the INPIONCH1 gene (such as the nucleic acid molecules whose sequences are presented in SEQ ID Nos: 3, 5, 7, 9, 11, 13, 15, 17 and 19, and fragments thereof).

Nucleic acid molecules of the invention may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridise to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (Methods Enzymol 1987. 152:399-407) and Kimmel, A. R. (Methods Enzymol. 1987 152:507-511).

“Stringency” refers to conditions in a hybridization reaction that favour the association of very similar molecules over association of molecules that differ. High stringency hybridization conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridization reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecules encoding the INPIONCH1 polypeptide (SEQ ID NO:2), and nucleic acid molecules that are substantially complementary to such nucleic acid molecules. Preferably, a nucleic acid molecule according to this aspect of the invention comprises a region that is at least 80% identical over its entire length to the nucleic acid molecule having the sequence given in SEQ ID NO:1, or a nucleic acid molecule that is complementary thereto. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the INPIONCH1, polypeptide.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INPIONCH1, polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INPIONCH1, polypeptide is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., Proc Natl Acad Sci USA 1988 85, 8998-9002). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridise with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, SM et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfested or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al.,[supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out tranription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego, Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk- or apr± cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison, Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Agonist and antagonist compounds or other modulatory compounds act by binding to either the pore forming region or the ligand binding domain of the polypeptides of the invention. Preferred candidate compounds for use in the screening methods described below may thus be compounds which have been identified as binding to the pore forming region or the ligand binding domain of the polypeptides of the first aspect of the invention.

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

The level of a signal may be detected by detecting a label, such as a fluorescent label, attached to the compound to be screened, or by any other means (for example a signal transmitted inside the cells.). Alternatively, the effect of the compound on ion influx through the polypeptide may be determined. For example, the cells expressing the polypeptide may be preloaded with ion sensitive dyes and their spectral response to the additional of the compound to be screened assessed (see Khan, N-A. J. Neuroimmunology, 1999, 99:53).

A further preferred method for identifying an agonist or antagonist of a polypeptide of the invention comprises:
(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises:

determining the inhibition of binding of a ligand to cells which have a polypeptide of the invention on the surface thereof, or to cell membranes containing such a polypeptide, or to a solid surface having such a polypeptide immobilized thereon, in the presence of a candidate compound under conditions to permit binding to the polypeptide, and determining the amount of ligand bound to the polypeptide. A compound capable of causing reduction of binding of a ligand is considered to be an agonist or antagonist Preferably the ligand is labelled. Competition assays of his type are described in detail in Example 2.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention, or any solid support on which a polypeptide of the invention is immobilized;

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments of the invention described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signalling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

Examples of suitable assays for the identification of agonists or antagonists of the polypeptides of the invention are described in Rosen et al., Curr. Opin. Drug Discov. Devel., 2003, 6(2):224-30.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy.

Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective-amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmfull to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intraarterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L. (Curr. Top. Microbiol. Inmunol., (1992) 158, 39-66) or adeno-associated virus (AAV) vectors as described by Muzyczka, N. (Curr. Top. Microbiol. Immunol., (1992) 158, 97-129) and. U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, (1986) 324, 163-166; Bej, et al., Crit. Rev. Biochem. Molec. Biol., (1991) 26, 301-334; Birkenmeyer et al., J. Virol. Meth., (1991) 35, 117-126; Van Brunt, J., Bio/Technology, (1990) 8, 291-294) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;

wherein detection of levels of the hybrid complex in the patient sample that differ from levels of the hybrid complex in the control sample is indicative of disease.

For example, results presented herein suggest that INPIONCH1 expression is up-regulated in inflammatory bowel disease and down-regulated in psoriasis and rheumatoid arthritis. A method of diagnosing inflammatory bowel disease may therefore involve detecting an increase in the INPIONCH1 transcript or the INPIONCH1 polypeptide and a method of diagnosing psoriasis and rheumatoid arthritis may involve detecting a decrease in the INPIONCH1 transcript or the INPIONCH1 polypeptide compared to a control.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridising amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1 985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application W095/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly certain diseases including, but not limited to, nausea, vomiting, pain, eating disorders, alcoholism, psychosis, side effects of various anticancer therapies, irritable bowel syndrome, gastrointestinal related disorders, Alzheimer's disease, Parkinson's disease, Huntington's disease, cognitive disorders, behavioural disorders and phobias such as anxiety related illnesses and addiction, obsessive compulsive behaviour, memory and learning disorders, depression and panic disorders, asthma, inflammation, sexual dysfunction, disorders of the neuroendocrine and cardiovascular systems.

Such kits will also be of use in diagnosing diseases diseases associated T cells such as inflammatory bowel diseases (including Crohns disease and ulcerative colitis), multiple sclerosis, psoriasis, rheumatoid arthritis, thrombocytopenia, type I diabetes mellitus, asthma, atopic dermatitis, atopic rhinitis and conjunctivitis, diseases associated with T cell proliferation such as leukaemias, diseases associated with T-cell depletion such as HIV infection, chemotherapy and radiotherapy, and diseases where regulation of T cell activation is required, such as cancers, viral infections, bacterial infections (including tuberculosis) and fungal infections, or susceptibility to such diseases.

Various aspects and embodiments of the present invention will now be described in more detail by way of example, with particular reference to INPIONCH1 polypeptide.

It will be appreciated that modification of detail may be made without departing from the scope of the invention.

### BRIEF DESCRIPTION OF THE FIGURES

FIG. 1: BLASTP results for INPIONCH1 versus public protein database. Top 10 annotated BLASTP matches for INPIONCH1 against the National Center for Biotechnology Information Non-Redundant protein database and alignment to match sharing greatest sequence identity.

FIG. 2: Conserved Domain search for INPIONCH1 versus PFAM domain specific public database. CDD alignment of the neurotransmitter gated ligand binding domain (PFAM02931) and INPIONCH1 polypeptide sequence.

FIG. 3: MEMSAT transmembrane and signal peptide prediction of INPIONCH1. MEMSAT report for INPIONCH1 showing four predicted transmembrane spanning regions shown in bold text and underlined in the INPIONCH1 sequence, and shown graphically below as sequential peaks.

FIG. 4: Alignment of 5-HT3 subunits from mouse and human with INPIONCH1.

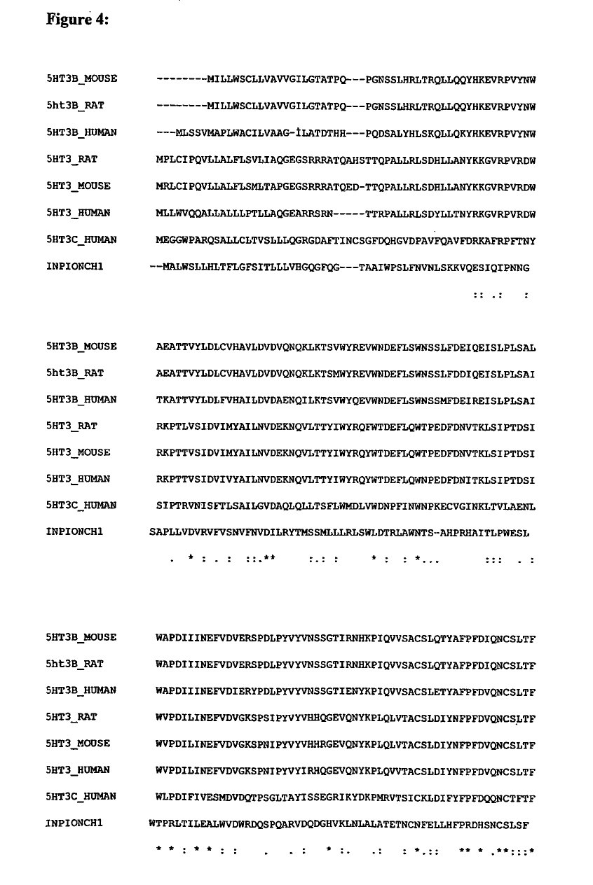

FIG. 5: Schematic depiction and sequence of the INPIONCH1 PCR primers.

FIG. 6: Tissue distribution of INPIONCH1. Amplification using the primers depicted was carried on the six human cDNAs. Arrows depict the 500 bp KIAA1067 fragment and the 275 bp the INPIONCH1 exons 4 and 5. The correctly spliced INPIONCH1 exons 4 and 5 could be detected only in thymus.

FIG. 7: PCR of the INPIONCH1 exons on human thymus and brain cDNA. Amplification of different exons was performed as described in the Examples. Arrows depict the 500 bp KIAA1067 fragment and the 275 bp the INPIONCH1 exons 4 and 5.

FIG. 8A: Tissue profile PCR of INPIONCH1. Primers INPIONCH1 Taq F and INPIONCH1 Taq R were used to amplify exon 5-6 boundary of INPIONCH1 from cDNA synthesized from RNA prepared from human cervix, pre-term placenta, neutrophils, embryo, foetal thymus and adult thymus. PCR products were separated on a 10% acrylamide gel. Lane M=Smart ladder small fragment (Eurogentec); lane 1=cervix; lane 2=pre-term placenta; lane 3=neutrophil; lane 4=embryo; lane 5=foetal thymus; lane 6=adult thymus; lane 7=genomic DNA; lane 8=NTC. +=reaction with reverse transcriptase; −=reaction without reverse transcriptase. The PCR product marked with an arrow is the spliced 91 bp product from INPIONCH1.

FIG. 8B: Expression of INPIONCH1 in Jurkat cells. Primers INPIONCH1 Taq F and INPIONCH01 Taq R were used to amplify exon 5-6 boundary of INPIONCH01 from cDNA synthesized from RNA prepared from untreated Jurkat cells and Jurkat cells activated with anti-CD3 and anti-CD28 for 2 hours. PCR products were separated on a 10% acrylamide gel. Lane M=Smart ladder small fragment (Eurogentec); lane 1=untreated Jurkat cells; lane 2=Jurkat cells activated for 2 hours. +=reaction with reverse transcriptase; −=reaction without reverse transcriptase. The PCR product marked with an arrow is the spliced 91bp product from INPIONCH0.

FIG. 9: Mammalian expression of the INPIONCH1. MCF-7 cells were transfected with either tagged INPIONCH1 or tagged 5HT3a cDNAs or a mixture of both. 48 hours following transfection, total cell lysate was prepared from the transfected and control cells, separated on an SDS gel and Western blotted with anti-V5 antibody.

FIG. 10: Schematic depiction of the ion channel chimeras and sequence of the primers.

FIG. 11: 5-HT induced current in HEK293 cells transfected with INPIONCH1. Current obtained from one cell in response to 10 sec application of 10 μM 5-HT.

FIG. 12: 5-HT induced current in HEK293 cells transfected with INPIONCH1. Current obtained from one cell in response to 10 sec application of 10 μM 5-HT.

FIG. 13: 5-HT induced current in HEK293 cells transfected with ion channel chimera, IS68. Current obtained from one cell in response to 10 sec application of 10 μM 5-HT. Following wash-out, 5-HT was applied a second time for 10 sec.

FIG. 14: 5-HT induced current in HEK293 cells transfected with ion channel chimera, IS67. Current obtained from one cell in response to 10 sec application of 10 μM 5-HT.

FIG. 15: Average normalized current (pA/pF) for 5HT-induced (10 μM) currents for the three different channels studied, INPIONCH1, IS67 and IS68.

FIG. 16: Current-voltage relationships for 5-HT-induced (10 mM) current following recording during voltage ramps (−90 to +90 mV).

FIG. 17: Tissue Profile PCR of INPIONCH1. Primers INPIONCH1 Taq F and INPIONCH1 Taq R were used to amplify exon 5-6 boundary of INPIONCH1 from cDNA synthesized from RNA prepared from human inflammatory bowel disease and clinically matched normal control RNA. PCR products were separated on a 10% acrylamide gel. Lane M=Smart ladder small fragment (Eurogentec); lane 1=inflammatory bowel disease; lane 2=clinically matched normal control. +=reaction with reverse transcriptase; −=reaction without reverse transcriptase. The PCR product marked with an arrow is the spliced 91 bp product from INPIONCH1.

FIG. 18: Tissue Profile PCR of INPIONCH1. Primers INPIONCH1 Taq F and INPIONCH1 Taq R were used to amplify exon 5-6 boundary of INPIONCH1 from cDNA synthesized from RNA prepared from psoriasis, rheumatoid arthritis and clinically matched normal control RNA. PCR products were separated on a 10% acrylamide gel. Lane M=Smart ladder small fragment (Eurogentec); lane 1=psoriasis; lane 2=psoriasis clinically matched normal control; lane 3=rheumatoid arthritis; lane 4=rheumatoid arthritis clinically matched normal control. +=reaction with reverse transcriptase; −=reaction without reverse transcriptase. The PCR product marked with an arrow is the spliced 91 bp product from INPIONCH1.

### EXAMPLES

### Example 1

### Identification of INPIONCH1

The polynucleotide and encoded polypeptide sequences representing INPIONCH1 (SEQ ID NO:1 and SEQ ID NO:2) are derived from an analysis of human genome sequence originating from chromosome 17q25. 1. The separate exons (both nucleic acid and protein) are presented in SEQ ID Nos: 3-20.

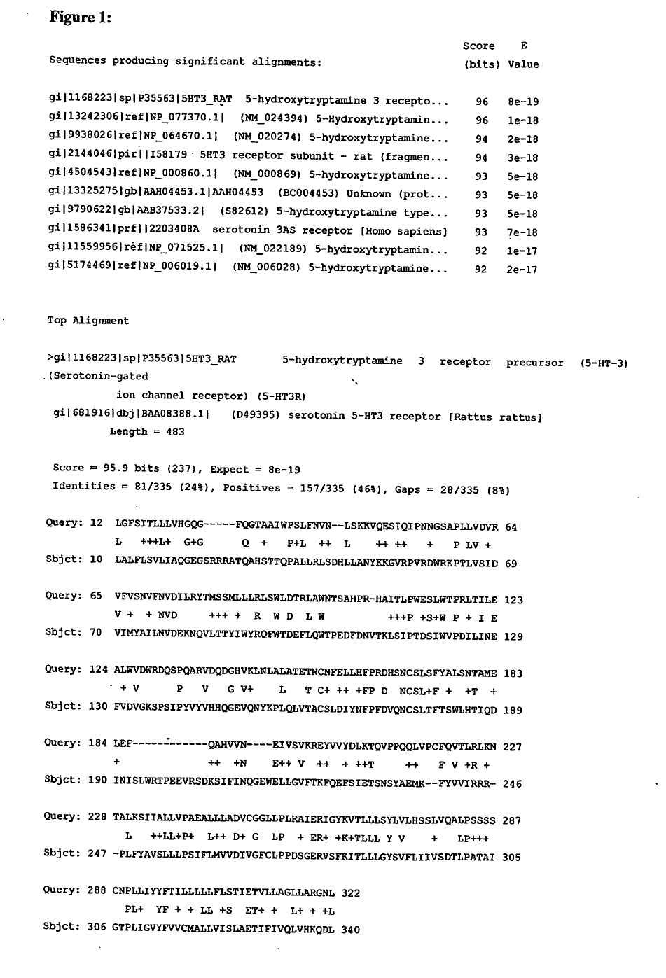

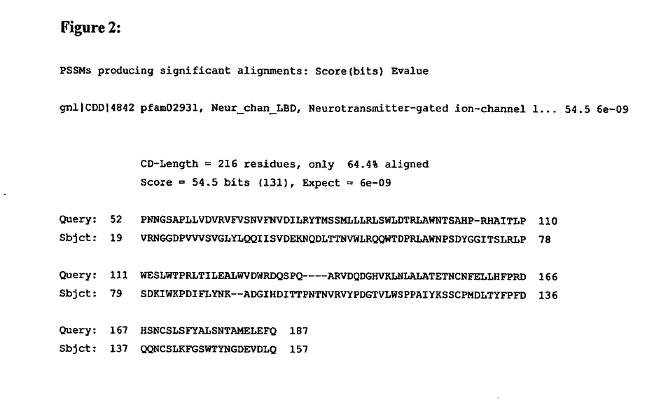

Based on BLAST? searches with the INPIONCH1 polypeptide sequence against public protein databases, this protein is most closely related to 5-HT3-b receptor subunit from the mouse, sharing 24% sequence identity in over the first 180 amino acids (FIG. 1). This region also aligns to the conserved PFAM (A. Bateman et al Nucleic Acids Research (2000) 28:263-266) ligand binding domain with a bit-wise alignment score of 60.5 (FIG. 2) using the CD-search programme (Altschul, SF., et al (1997) Nucleic Acids Res. 25:3389-3402.).

Comparison between the INPIONCH1 amino acid sequence and the newly crystallised ligand binding domain of the acetylcholine receptor channel (ACHR) over the first 200 amino acids revealed an alignment confidence of 96%. The structure of the ACHR ligand binding domain is thought to represent a common structural architecture adopted by all the ligand gated ion channels.

Furthermore, the polypeptide sequence representing INPIONCH1 is predicted to contain 4 transmembrane spanning regions, based on the MEMSAT prediction programme (Jones, D. T., et al. (1994) Biochemistry. 33:3038-3049). Both this programme and SignalP signal peptide prediction tool identified a signal peptide at the beginning of the INPIONCH1 sequence in the first 25 amino acids. The SignalP programme incorporates both neural network-based and hidden Markov model-based methods to enhance its predictive power (Nielsen H, Protein Engineering (1997) 10, 1-6). The signal peptide consists of amino acid residues 1-23 with the mature peptide starting at residue 24.

The polynucleotide sequence encoding the INPIONCH1 protein sequence presented in SEQ ID NO:2 is composed of 9 exons which are of similar length and content, with respect to domain and secondary structural elements, to the conserved exon structure found in human and murine 5-HT3 subunits (Bruss et al Neuropharmacology (2000) 39: 308-315). Full length cDNA sequence (SEQ ID NO:1—cDNA sequence) representing exons 1 through 9 has been cloned from thymus and brain tissue libraries. This sequence represents exactly the fill length INPIONCH1 DNA sequence (SEQ ID NO: 1).

No orthologue of INPIONCH1 was identified in rodents. However, a fragment having 80% sequence identity to exons 4 and 5 of INPIONCH1 was identified on mouse chromosome 11, suggesting the existence of a rodent pseudogene. This unusual difference between humans and rodents points to the polypeptide being particularly useful in the regulation of human physiology and pathophysiology.

### Example 2

### Cloning of INPIONCH1

Tissue Distribution Analysis

A. Generation of cDNA from Human Tissue RNA 1 μg total RNA (Ambion) from different human tissues was used to generate cDNA using the Thermoscript RT (Invitrogen) and oligo dT primer following the manufacturer's protocol. The reaction was incubated for 20 min at each of the temperatures: 50° C., 55° C. and 65° C. and 2 μl of the reaction were used in the PCR.

B. PCR for Tissue Distribution of INPIONCH1

The initial approach for assessing tissue distribution of the INPIONCH1 was the PCR amplification of exon 5, which proved to be the least conserved in the alignment of the ion channel family of proteins.

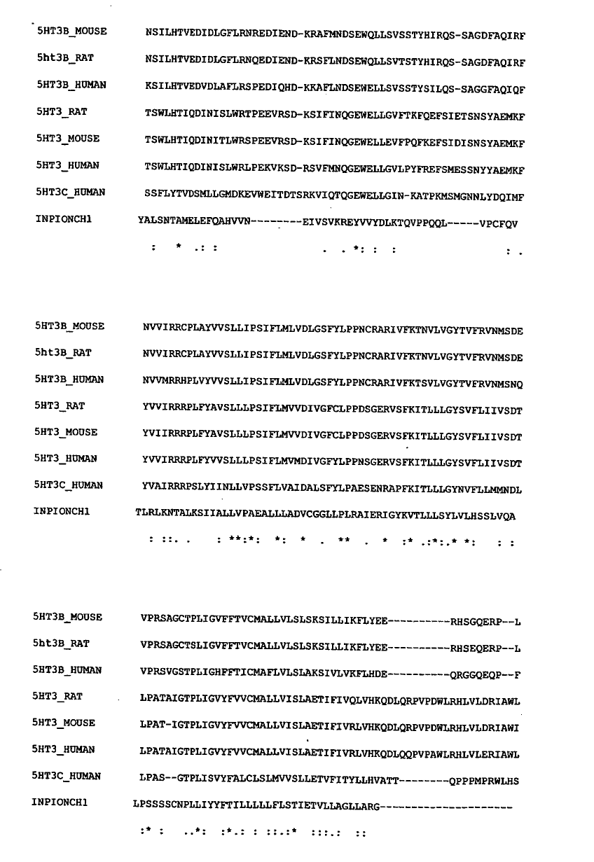

Primers IC1.1 and IC1.2 (FIG. 5) were used to amplify exon 5 of the INPIONCH1 in the following human cDNAs: adrenal, cervix, liver, lung, heart, prostate, skeletal muscle, small intestine, spleen, stomach, testis, thymus and brain and placenta. Exon 5 appeared to be expressed in the majority of tissues tested.

However, we suspected that primers IC1.1 and IC1.2 might amplify a sequence derived from mRNA that is transcribed in the opposite direction to the INPIONCH1 mRNA, belonging to a gene termed KIAA1067. Transcripts of the KIAA1067 gene are ubiquitous and abundant in most human tissues. We appreciated that the 3′-untranslated region of the KIAA1067 mRNA is complementary to both part of the protein-coding and 3′-untranslated regions of the INPIONCH1 gene. The available ESTs belong to the KIAA1067 clone and contain the intron sequences of INPIONCH1. Based on the EST data, the 3′-untranslated region of the KIAA1067 clone is expected to extend up to the region complementary to the intron region between exons 4 and 5 of the INPIONCH1 sequence.

Thus, primers designed to exon 5 of INPIONCH1 will amplify both the ion channel and KIAA1067 as the mRNA species for these two clones in this region are complementary.

Primers IC1.5 and IC1.2 were specifically designed to amplify only INPIONCH1 based on the known extent of KIAA1067. Thus, primer IC1.5 was designed in exon 4 of INPIONCH1 and is predicted to lie beyond the known sequence of KIAA1067. Primer IC1.2 is located in exon 5.

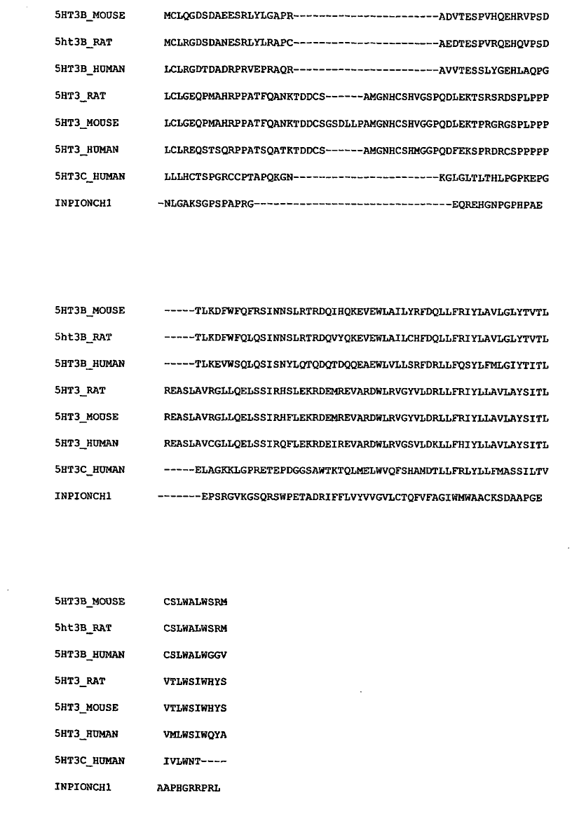

PCR reactions were completed using these two primers from the same array of tissues described above. Surprisingly, two PCR products were obtained. The most abundant PCR fragment was ˜500 bp and appeared to be the unspliced transcript (FIG. 6). This PCR product was subcloned into pGEMTEasy (Promega) and sequenced. The sequence confirmed that the product was the unspliced transcript consisting of exons 4 and 5 together with the intervening intron of INPIONCH1.

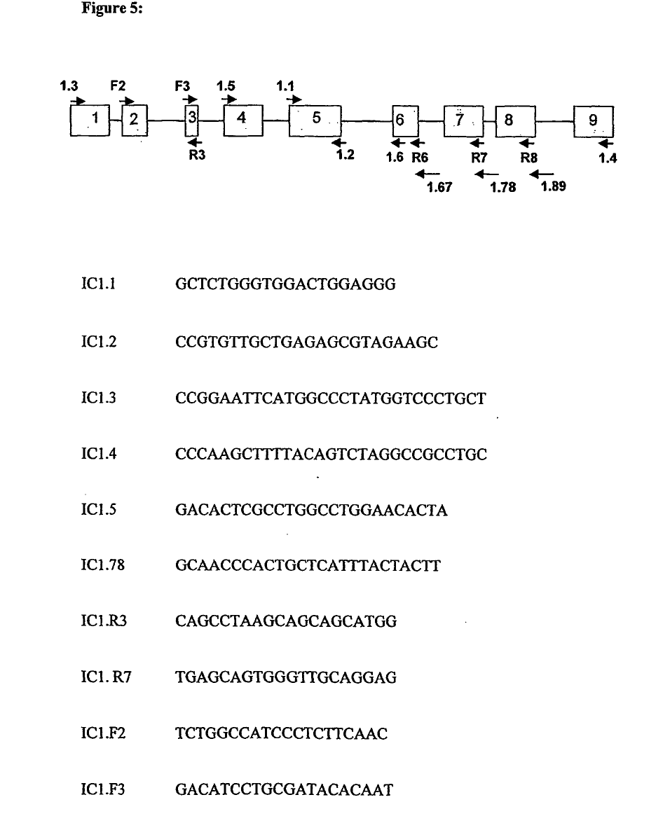

The correctly spliced transcript comprising nucleotides from exons 4 and 5 (without nucleotides from the intervening intron) could be detected only in thymus and brain (FIG. 7). This PCR product was sequenced and demonstrated to be the correctly spliced product corresponding to the predicted INPIONCH1 gene.

These data indicate that the 3′UT of KIAA1067 extends beyond the known sequence. In order to avoid the amplification of the KIAA1067 gene during cloning and to profile the INPIONCH1 tissue expression correctly, we designed a series of primers upstream and downstream of exons 4 and 5 (FIG. 5). Primers downstream of exon 5 cross the exon-exon boundaries to maximise the amplification of the correctly spliced INPIONCH1 cDNA and to circumvent generation of KIAA1067 fragments.

PCR amplification was completed using the reverse primer IC1.2 together with primers designed in exon 2 (IC.F2) and exon 3 (IC.F3) of the INPIONCH1 gene. Exon 2 and exon 3 were not thought to be complementary to the KLAA1067 sequence. However, surprisingly, abundant PCR products were obtained that corresponded to the unspliced mRNA of KIAA1067. This data indicates that the 3′UT of the KIAA1067 clone extends beyond the available ESTs (FIG. 7). Therefore, extreme caution is required in the design of primer pairs to visualise specifically INPIONCH1 and not KIAA1067. Any primer pairs designed within a single exon of INPIONCH1 will be unable to distinguish between transcripts of INPIONCH1 and KIAA1067, and, due to the ubiquitous expression of KIAA1067, will amplify this gene. It is only using our bespoke cloning exercise that INPIONCH1 is distinguishable.

In order to attempt to quantify the expression profile of INPIONCH1 further, a Taqman primer probe set was designed over the boundary of exons 5-6. This primer-probe set was analysed by BLAST® (Basic Local Aligmnent Search Tool, Altschul SF, Gish W, Miller W, Myers E W, Lipman D J.: J Mol Biol Oct. 5, 1990 ;215(3):403-10). Results confirmed that each oligonucleotide recognises the target sequence with a specificity >3 bp when compared to other known cDNAs or genomic sequence represented in the Unigene and GoldenPath publicly available databases.

The sequence of the primers and probe are:

| | |
|---|---|
| INPIONCH1 Taq F | CAACTGCAGCCTCAGCTTCTAC |
| INPIONCH1 Taq P | CTCAGCAACACGGCGATGGAGTTAGAGTT |
| INPIONCH1 Taq R | TCTTGACACTCACAATCTCGTTCA |

This primer probe set would amplify a fragment of 91bp for the spliced INPIONCH1 gene product, and 944 bp for the unspliced KIAA1067 transcript-containing intron sequence. The expression of INPIONCH1 was found to be too low for a significant signal to be generated for quantitation by real-time PCR. Therefore, PCR products generated by using these forward and reverse primers were analysed by electrophoresis on a 10% acrylamide gel.

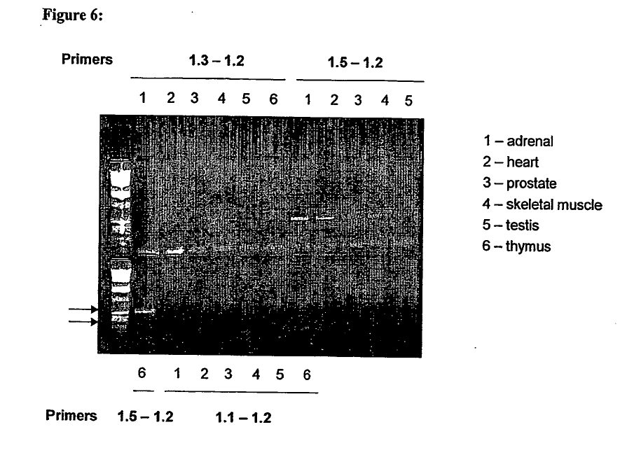

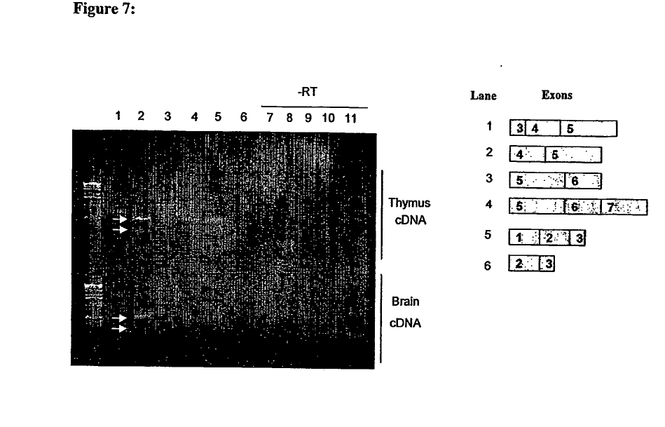

As shown in FIG. 8A, a 91 bp product was seen in both +RT and −RT samples from all tissues, but not in the genomic DNA control. The unspliced PCR fragment was only seen in the genomic DNA control. For foetal thymus, adult thymus and possibly pre-term placenta, the signal of the 91 bp fragment is significantly higher in the +RT samples compared to the −RT samples.

As shown in FIG. 8B, a 91 bp product was seen in the RT+ sample in RNA extracted from Jurkat cells, a human T cell line (resting and after stimulation with anti-CD3).

In conclusion, our experiments suggest that while most human tissues contain the KIAA1067 clone, the expression of the INPIONCH1 is unexpectedly low and localised mostly in human thymus, brain and T cells.

C. Cloning the Full-Length INPIONCH1

Cloning of the INPIONCH1 was performed using human thymus cDNA and the following PCR conditions: DyNAzyme EXT DNA Polymerase (Finnzymes), 2 mM MgCl2, 5% DMSO. The annealing temperature varied between 55° C.-65° C. depending on the primers used.

Due to the difficulty in amplifying the full-length molecule, the following amplification reactions were set in order to obtain the INPIONCH1 fragments:

First round of PCR:

| Primers (Appendix 1) | Template | Product |
|---|---|---|
| IC1.3 + ICR.3 | cDNA | Exons 1-3 |
| ICF.3 + IC1.2 | cDNA | Exons 3-5 |
| IC1.1 + ICR.7 | cDNA | Exons 5-7 |
| IC1.78 + IC1.4 | cDNA | Exons 8-9 |

The PCR products were directly cloned into pGEM-Teasy vector (Promega) and sequenced using T7 and SP6 primers. The sequences of the fragments were identical to those for the predicted INPIONCH1 sequence.

A second round of PCR was employed to assemble the INPIONCH1 cDNA. The template was a 1:1 mixture of the two purified fragments:

| Primers (Appendix 1) | Template | Product |
|---|---|---|
| IC1.3 + IC1.2 | Ex 1-3 + Ex 3-5 | Exons 1-5 |
| IC1.1 + IC1.4 | Ex 5-7 + Ex 8-9* | Exons 5-9 |

*Ex 5-7 and Ex 8-9 contain a 20-mer overlap due to the amplification of Ex 8-9 using primer IC1.78 that spans the junction of exons 7 and 8.

The PCR products were directly cloned into pGEM-Teasy vector (Promega) and sequenced using T7 and SP6 primers. The sequences of the fragments and the full-length INPIONCH1 cDNA confirmed the gene prediction.

D. Expression of the INPIONCH1 in Mammalian Cells

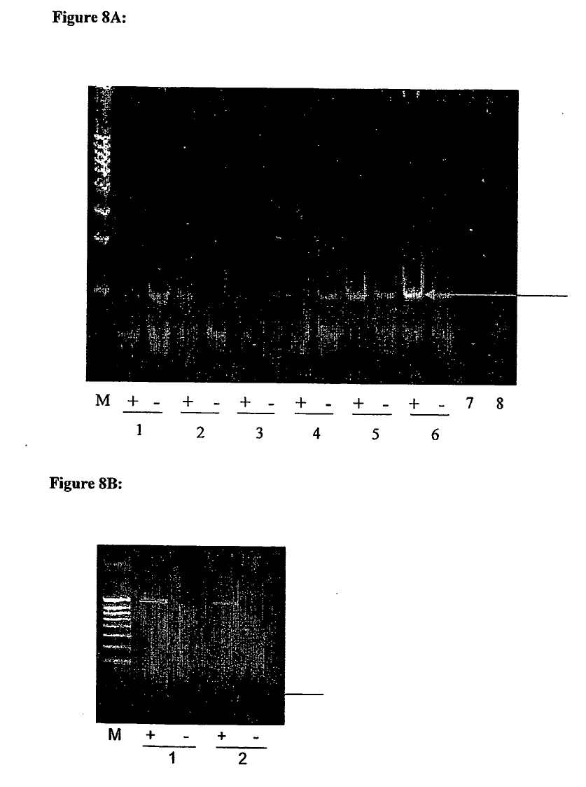

The INPIONCH1 cDNA was cloned into the pcDNA3.1 V5/His TOPO expression vector (invitrogen). The construct was transfected into different cell line and the expression was monitored by Western Blot using the anti-V5 antibody (FIG. 9).

E. Construction of Ion Channel Chimeras

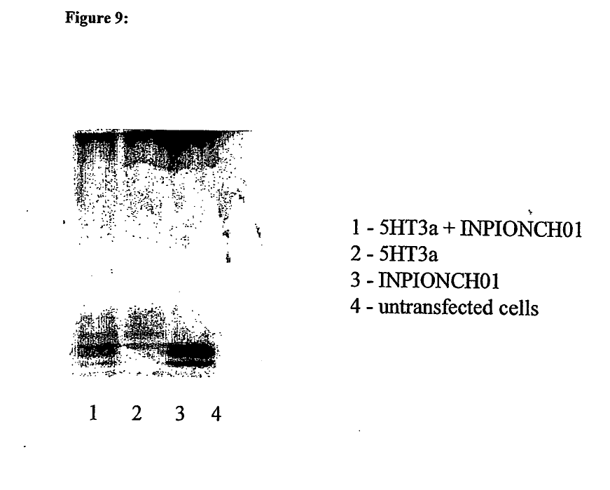

The INPIONCH1 protein displays all of the structural motifs that are common to the superfamily of Cys loop ligand-gated ion channels. However, due to its low sequence identity with other receptor members, it may also bind a novel class of ligands. In order to study the INPIONCH1 function in the absence of a ligand, we used the 5HT3a channel to construct two ion channel chimeras for INPIONCH1. The ligand binding domains (LBD) and the transmembrane (TM) domains of INPIONCH1 and human 5HT3a were amplified by PCR and a unique EcoRI site was introduced between the LBD and the TM domain. PCR fragments were digested with the respective restriction sites (LBD with HindIII and EcoRI; TM with EcoRI and XbaI) and subcloned into pBluescript pKS+ vector (Stratagene). Using the EcoRI restriction site, the fragments were joined in order to form the two chimeras: 5HT3a/INPIONCH1 and INPIONCH1/5HT3a (FIG. 10). Following cloning and sequencing into pKS+ vectors the cDNAs were cloned into the mammalian expression vectors pcDNA3.1-V5/His TOPO (Invitrogen).

### Example 3

### Identification of Ligands of INPIONCH1

A. Binding of a Radiolabelled Ligand to the Ion Channel and Competition Assay

Binding assays were performed in glass filter 96 well plates in which either cells or cell membranes expressing the ion channel were incubated in the presence of increasing concentrations of the radiolabelled ligand together with (non-specific binding) or without (total binding) unlabeled ligand. Following association, excess ligand was washed and dried filters were counted in a scintillation counter. Specific binding was measured by subtracting non-specific counts from the total binding. The dissociation constant (Kd) and the receptor density (Bmax) were calculated according to Scatchard calculations using GraphPad software.

In order to determine if other compounds bind to the ion channel, a competition assay was set up by incubation of the cells or cell membranes in the presence of radiolabelled ligand and unlabelled compounds. A decrease in the specific binding of the radiolabelled ligand indicates the presence of a competitor.

B. Binding of a Fluorescent Ligand to the Ion Channel—Fluorescence Polarization Method

Binding of a fluorescent ligand to cells or cell membranes expressing the ion channel can be monitored directly by fluorescent polarization (FP). Following excitation with plane-polarized light, fluorescent molecules emit light in the same plane assuming that the molecule remains stationary. The degree to which emission intensity moves from one plane to another is related to the mobility of the labelled molecule. In turn, the mobility is directly proportional to the receptor binding. (Chipperton, P. Curr. Drug Discovery, September 2001, page 17).

This assay can be adapted to identify small molecule competitors for the ligand binding site.

This assay uses the novel tagged ion channel ligand-binding domain (LBD) and a selective fluorescent ligand in a homogenous mix-and-read assay format. Ion channel LBD is added to the fluorescent ligand to form an ion channel-LBD/fluorescent ligand complex resulting in a high polarization value. For the competition experiments this complex is then added to individual test compounds in microwell plates. Competitors displace the fluorescent ligand from the ion channel-LBD, causing the fluorescent ligand to tumble rapidly during its fluorescence lifetime, resulting in a low polarization value. Noncompetitors will not displace the fluorescent ligand from the complex, so the polarization value remains high. The shift in polarization value in the presence of test compounds is used to determine relative affinity of test compounds for ion channel-LBD.

B. Monitoring Ion Influx into Cells Expressing INPIONCH1 or the Chimeras

Agonists of the ion channel induce ion influx into cells. In order to monitor these changes, cells expressing INPIONCH1 were preloaded with ion sensitive dyes (for example, sodium-sensitive SBFI and potassium-sensitive PBFI) and their spectral responses upon addition of compounds were measured (Khan, N-A and Poisson J-P, J. Neuroimmunology, 99:53, 1999).

### Example 4

### Electrophysiology Studies

The whole cell patch-clamp technique was used to record currents from HEK293 cells transfected with either INPIONCH1 or one of the chimeras 5HT3a/INPIONCH1 (IS67) and INPIONCH1/5HT3a (IS68). The buffer for the bath was 140 mM NaCl, 4.7 mM KCl, 1.2 mM MgCl₂, 2.5 mM CaCl₂, 21 mM HEPES pH 7.4. The pipette buffer was 140 mM KCl, 2 mM MgCl₂, 11 mM EGTA, 10 mM HEPES pH 7.4. In some experiments, HEPES in the bath was replaced by Tricene but this had no effect on the currents. Cells were clamped at −80 mV or −60 mV. Drugs were applied by pressure ejection from modified micropipettes. Currents were recorded using an Axopatch 1A amplified (Axon Instruments) and measured and analysed using Pclamp 6 (Axon Instruments) and Origin software.

Initial experiments were completed on cells following transient expression of the channel, INPIONCH1. However, under these circumstances, no reproducible currents could be measured. This caused difficulties in analysing results. Under these conditions, the cells appeared to possess a variable “leak” current and this further obscured analysis of any ligand gated channel function.

To overcome these difficulties, HEK293 cells were transfected with either INPIONCH1 or one of the two chimeras (IS67 or IS68) and subsequently placed under selection pressure. Following transfection, positively expressing cells were selected by treatment with 1 mM neomycin and only cells that grew under these conditions were used to determine the electrophysiological characteristics of the various expressed clones. Only a very small proportion of cells grew under these conditions.

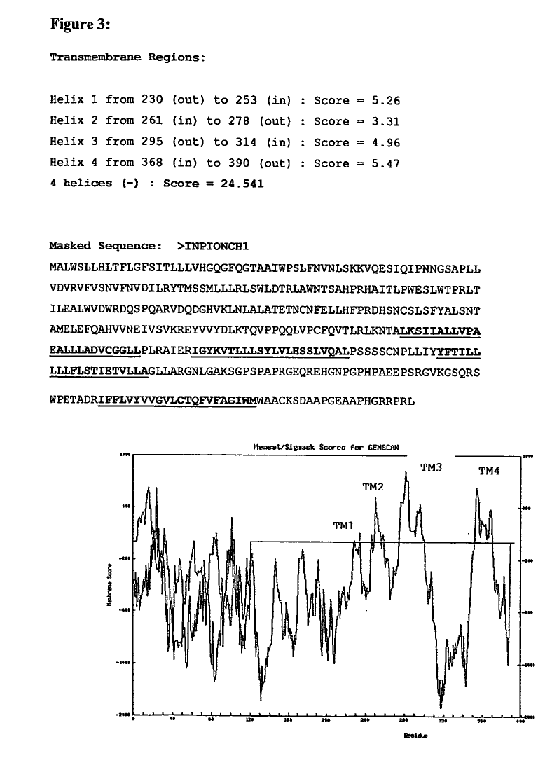

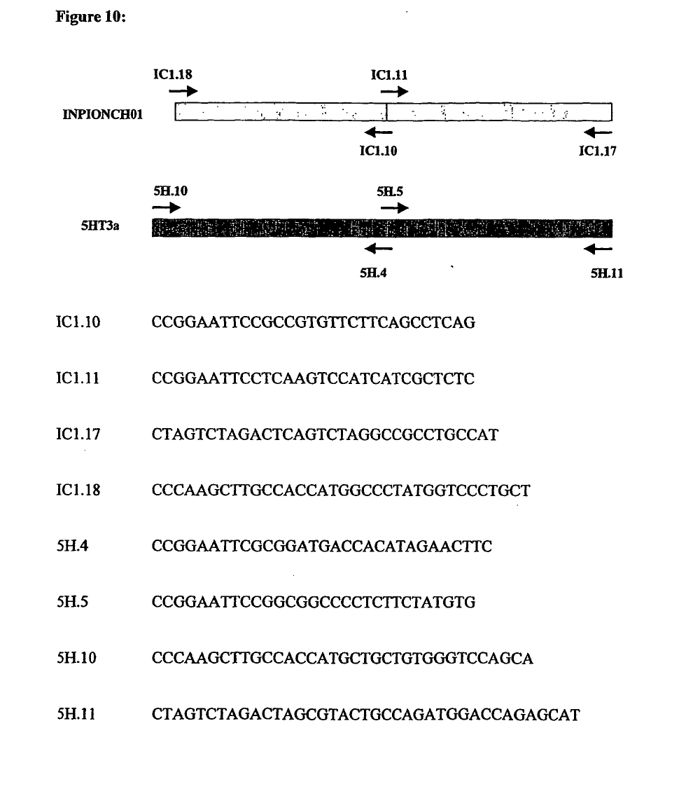

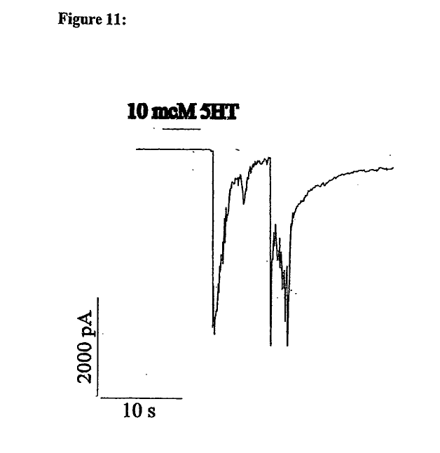

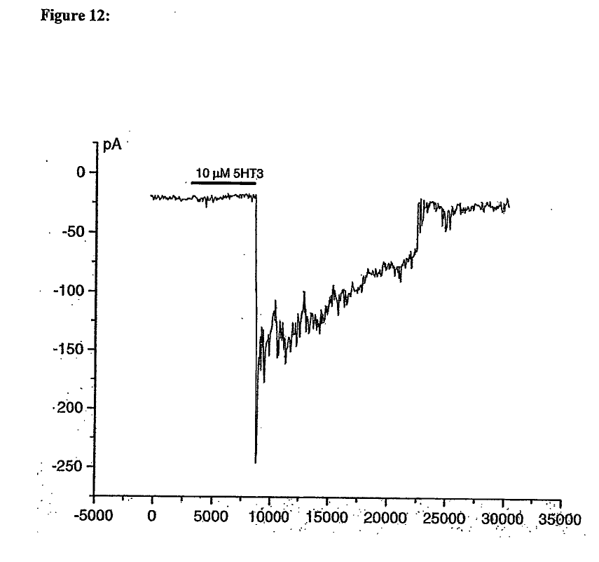

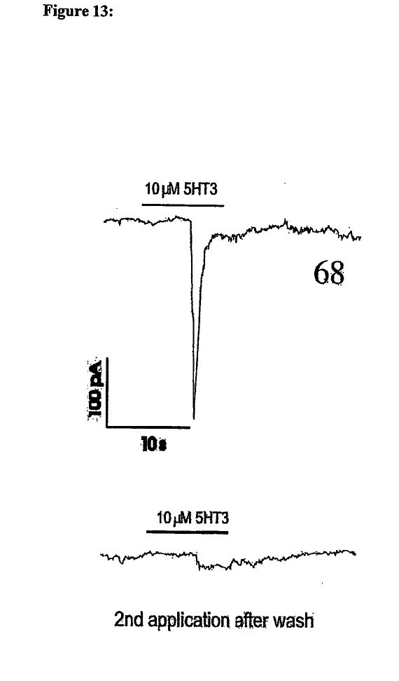

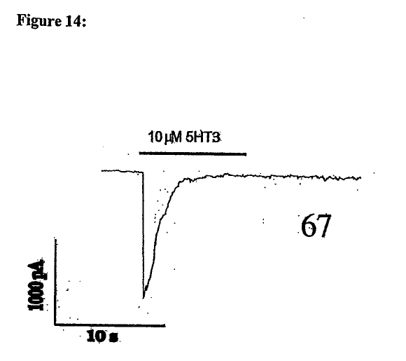

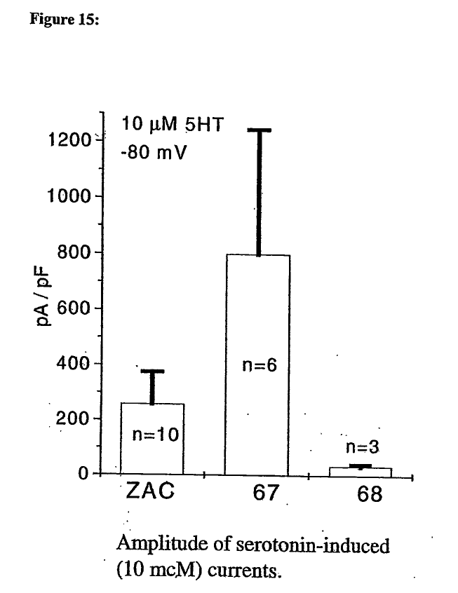

Application of 5-HT (serotonin) (10 μM) to HEK293 cells expressing INPIONCH1 resulted in an inward current (FIGS. 11 and 12). The nature of this current varied dependent on the efficiency of the 5-HT wash-out from the bath. Similar currents in response to 5-HT were also observed in cells transfected with either of the ion channel chimeras, IS67 and IS68 (FIGS. 13, 14 and 15). The channels were easily desensitised to 5-HT; thus, the response was minimal or absent in cells following a second application of 5-HT (FIG. 3). Voltage ramps (−90 to +90 mV) were applied to cells expressing INPIONCH1 before and after 5-HT application and the current—voltage relationships analysed. This demonstrated that the reversal potential was about 0 mV indicating that the channel is acting as a non-selective cation channel (FIG. 16). Tubocurarine appeared to have no effect on either the leak channel or the 5-HT induced channel.

Application of acteylcholine, nicotine or noradrenaline appeared not to activate current in cells transfected with INPIONCH1. However, in some cells, nicotine appeared to block outward current recorded at positive potentials suggesting that it could have antagonist actions. This result suggests that INPIONCH1 may form homopentamers with subunits of other ligand-gated ion channels. This is consistent with the known assembly of ligand gated ion channels such that family members can homo- and hetero-pentamerise to form the ligand binding pocket and the channel.

The effect of zinc on the channel was measured. Application of zinc sulphate (1 mM) appeared to be toxic to the cells. However, no consistent specific zinc activated current was observed. Application of zinc sulphate to the cells appeared to reduce the amplitude of the current in response to 5-HT. However, 5-HT was able to induce currents before and after the application of zinc sulphate (1 mM).

In conclusion, heterologous expression of INPIONCH1 in HEK2093 cells confers 5-HT ligand gated channel activity on the cells that is not present in untransfected cells. The two chimeras behave in a similar fashion. The channel acts as a non-selective cation channel and under normal physiological conditions, activation of this channel will result in the influx of sodium into the cell.

### Example 6

### Tissue Distribution Analysis

A. Generation of cDNA from Human Tissue RNA

500 ng total RNA (Clinomics, Mass., USA) from different diseased and clinically matched normal human tissues was used to generate cDNA using the Thermoscript reverse transcriptase (Invitrogen) and oligo dT primer following the manufacturer's protocol. The reaction was sequentially incubated for 4 min 30 sec at each degree C of a temperature gradient from 50-60° C. and the reverse transcriptase then deactivated by incubating at 85° C. 5 min. 4 μl of the reaction was used in the PCR.

B. PCR for tissue distribution of INPIONCH1

In order to attempt to quantify the expression profile of INPIONCH1 in disease tissues, a Taqman primer probe set was designed over the boundary of exons 5-6. This primer-probe set was analysed by BLAST® (Basic Local Alignment Search Tool, Altschul SF, Gish W, Miller W, Myers E W, Lipman D J.: J Mol Biol Oct. 5, 1990;215(3):403-10). Results confirmed that each oligonucleotide recognises the target sequence with a specificity >3 bp when compared to other known cDNAs or genomic sequence represented in the Unigene and GoldenPath publicly available databases.

The sequence of the primers and probe are:

| | |
|---|---|
| INPIONCH1 Taq F | CAACTGCAGCCTCAGCTTCTAC |
| INPIONCH1 Taq P | CTCAGCAACACGGCGATGGAGTTAGAGTT |
| INPIONCH1 Taq R | TCTTGACACTCACAATCTCGTTCA |

This primer probe set would amplify a fragment of 91 bp for the spliced INPIONCH1 gene product, and 944 bp for the unspliced KIAA1067 transcript containing intron sequence. The expression of INPIONCH1 was found to be too low for a significant signal to be generated for quantitation by real-time PCR. Therefore, PCR products generated by using these forward and reverse primers were analysed by electrophoresis on a 10% acrylamide gel.

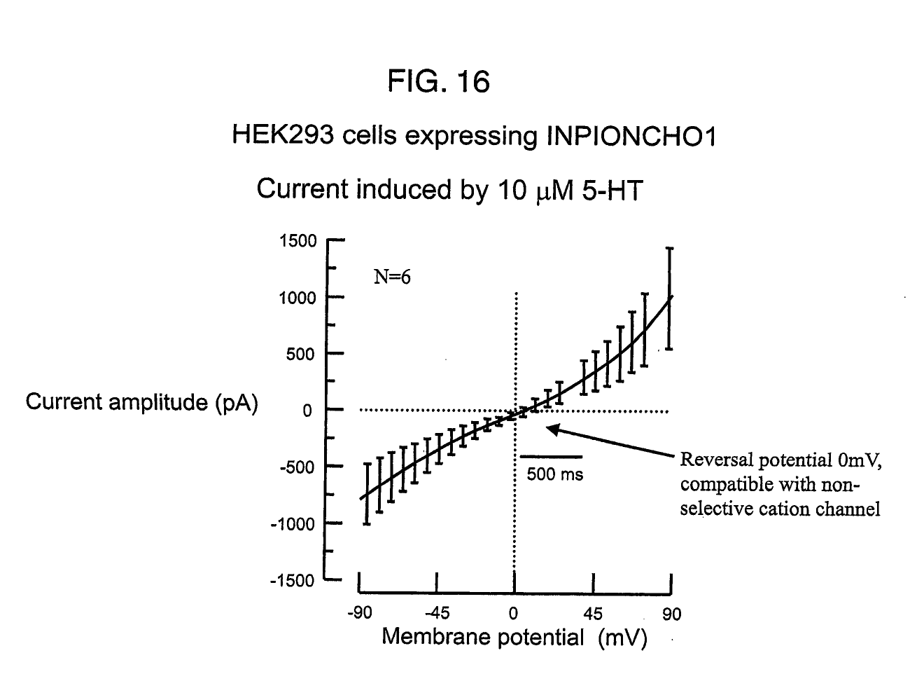

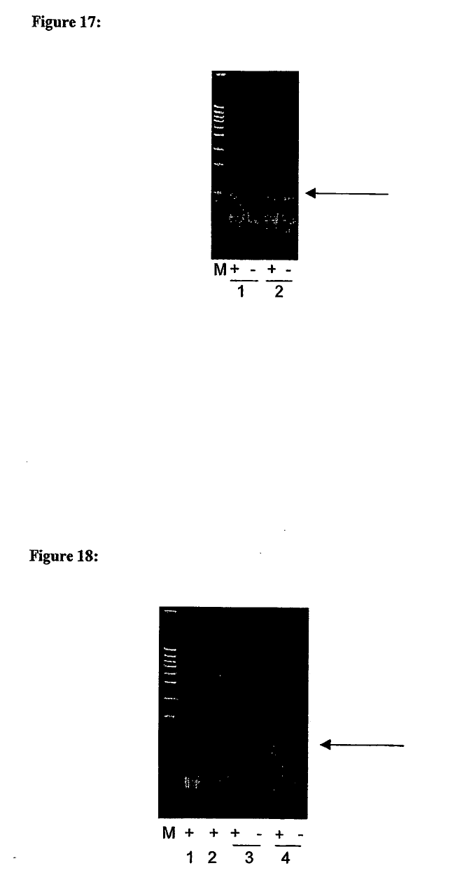

As shown in FIG. 17, a 91 bp product was seen in both +RT and −RT samples from clinically matched normal tissue for IBD, whereas a signal was seen only in the +RT sample for the IBD sample. This suggests that the transcript of INPIONCH1 is up-regulated in IBD compared to clinically matched normal tissue. As shown in FIG. 18, a 91 bp product was seen in the +RT sample for psoriasis clinically matched normal control, but not in the +RT sample for psoriasis. Also in FIG. 18, a 91 bp product was seen in both +RT and −RT samples from clinically matched normal tissue for rheumatoid arthritis, with the product being stronger in the +RT sample. For the rheumatoid arthritis sample, only a faint product could be seen in the −RT sample. Therefore, these results suggest that INPIONCH1 expression is down-regulated in psoriasis and rheumatoid arthritis.

| List of INPIONCH1-specific sequences: | |
|---|---|
| SEQ ID 1: Full length cloned cDNA sequence for INPIONCH1 | |
| 1 | ATGGCCCTAT GGTCCCTGCT CCATCTCACC TTCCTGGGGT TCAGCATTAC CTTGCTGTTG |
| 61 | GTCCACGGGC AGGGCTTCCA AGGGACAGCA GCCATCTGGC CATCCCTCTT CAACGTCAAC |
| 121 | TTGTCCAAGA AGGTTCAGGA AAGCATCCAG ATCCCGAACA ATGGGAGTGC GCCCCTGCTC |
| 181 | GTGGATGTGC GGGTGTTTGT CTCCAACGTG TTTAATGTGG ACATCCTGCG ATACACAATG |
| 241 | TCCTCCATGC TGCTGCTTAG GCTGTCCTGG CTGGACACTC GCCTGGCCTG GAACACTAGT |
| 301 | GCACACCCGC GGCACGCCAT CACGCTGCCC TGGGAGTCTC TCTGGACACC AAGGCTCACC |
| 361 | ATCCTGGAGG CGCTCTGGGT GGACTGGAGG GACCAGAGCC CCCAGGCTCG AGTAGACCAG |
| 421 | GACGGCCACG TGAAGCTCAA CCTGGCCCTC GCCACGGAGA CCAACTGCAA CTTTGAGCTC |
| 481 | CTCCACTTCC CCCGGGACCA CAGCAACTGC AGCCTCAGCT TCTACGCTCT CAGCAACACG |
| 541 | GCGATGGAGT TAGAGTTCCA GGCCCACGTG GTGAACGAGA TTGTGAGTGT CAAGAGGGAA |
| 601 | TACGTAGTTT ATGATCTGAA GACCCAAGTC CCACCCCAGC AGCTGGTGCC CTGCTTCCAG |
| 661 | GTGACGCTGA GGCTGAAGAA CACGGCGCTC AAGTCCATCA TCGCTCTCTT GGTGCCTGCA |
| 721 | GAGGCACTGC TGTTGGCTGA CGTGTGCGGG GGGTTGCTGC CCCTCCGGGC CATTGAGCGC |
| 781 | ATAGGCTACA AGGTGACATT GCTGCTGAGT TACCTCGTCC TCCACTCCTC CCTGGTGCAG |
| 841 | GCCCTGCCCA GCTCCTCCTC CTGCAACCCA CTGCTCATTT ACTACTTCAC CATCCTGCTG |
| 901 | CTGCTGCTCT TCCTCAGCAC CATAGAGACT GTGCTGCTGG CTGGGCTGCT GGCCCGGGGC |
| 961 | AACCTTGGGG CCAAGAGCGG CCCCAGCCCA GCCCCGAGAG GGGAACAGCG AGAGCACGGC |
| 1021 | AACCCAGGGC CTCATCCTGC TGAAGAGCCC TCCAGAGGAG TAAAGGGGTC ACAGAGAAGC |
| 1081 | TGGCCTGAGA CTGCTGACCG CATCTTCTTC CTCGTGTATG TGGTTGGGGT GCTGTGCACC |
| 1141 | CAATTCGTCT TTGCAGGAAT CTGGATGTGG GCAGCGTGCA AGTCTGACGC AGCCCCTGGA |
| 1201 | GAGGCTGCAC CCCATGGCAG GCGGCCTAGA CTGTAA |
| SEQ ID 2: INPIONCH1 amino acid sequence | |
| 1 | MALWSLLHLT FLGFSITLLL VHGQGFQGTA AIWPSLFNVN LSKKVQESIQ IPNNGSAPLL |
| 61 | VDVRVFVSNV FNVDILRYTM SSMLLLRLSW LDTRLAWNTS AHPRHAITLP WESLWTPRLT |
| 121 | ILEALWVDWR DQSPQARVDQ DGHVKLNLAL ATETNCNFEL LHFPRDHSNC SLSFYALSNT |
| 181 | AMELEFQAHV VNEIVSVKRE YVVYDLKTQV PPQQLVPCFQ VTLRLKNTAL KSIIALLVPA |
| 241 | EALLLADVCG GLLPLRAIER IGYKVTLLLS YLVLHSSLVQ ALPSSSSCNP LLIYYFTILL |
| 301 | LLLFLSTIET VLLAGLLARG NLGAKSGPSP APRGEQREHG NPGPHPAEEP SRGVKGSQRS |
| 361 | WPETADRIFF LVYVVGVLCT QFVFAGIWNW AACKSDAAPG EAAPHGRRPR L |
| SEQ ID 3: INPIONCH1 exon 1 DNA sequence | |
| 1 | ATGGCCCTAT GGTCCCTGCT CCATCTCACC TTCCTGGGGT TCAGCATTAC CTTGCTGTTG |
| 61 | GTCCACGGGC AGGGCTTCCA AGGGACAGCA GCCA |
| SEQ ID 4: INPIONCH1 exon 1 amino acid sequence | |
| 1 | MALWSLLHLT FLGFSITLLL VHGQGFQGTA AI |
| SEQ ID 5: INPIONCH1 exon 2 DNA sequence | |
| 1 | TCTGGCCATC CCTCTTCAAC GTCAACTTGT CCAAGAAGGT TCAGGAAAGC ATCCAGATCC |
| 61 | CGAACAATGG GAGTGCGCCC CTGCTCGTGG ATGTGCGGGT GTTTGTCTCC AACGTGTTTA |
| 121 | ATGTG |
| SEQ ID 6: INPIONCH1 exon 2 amino acid sequence | |
| 1 | WPSLFNVNLS KKVQESIQIP NNGSAPLLVD VRVFVSNVFN V |
| SEQ ID 7: INPIONCH1 exon 3 DNA sequence | |
| 1 | GACATCCTGC GATACACAAT GTCCTCCATG CTGCTGCTTA GGCTG |
| SEQ ID 8: INPIONCH1 exon 3 amino acid sequence | |
| 1 | DILRYTMSSM LLLRL |
| SEQ ID 9: INPIONCH1 exon 4 DNA sequence | |
| 1 | TCCTGGCTGG ACACTCGCCT GGCCTGGAAC ACTAGTGCAC ACCCGCGGCA CGCCATCACG |
| 61 | CTGCCCTGGG AGTCTCTCTG GACACCAAGG CTCACCATCC TGGAGGC |
| SEQ ID 10: INPIONCH1 exon 4 amino acid sequence | |
| 1 | SWLDTRLAWN TSAHPRHAIT LPWESLWTPR LTILEA |
| SEQ ID 11: INPIONCH1 exon 5 DNA sequence | |
| 1 | GCTCTGGGTG GACTGGAGGG ACCAGAGCCC CCAGGCTCGA GTAGACCAGG ACGGCCACGT |
| 61 | GAAGCTCAAC CTGGCCCTCG CCACGGAGAC CAACTGCAAC TTTGAGCTCC TCCACTTCCC |
| 121 | CCGGGACCAC AGCAACTGCA GCCTCAGCTT CTACGCTCTC AGCAACACGG |
| SEQ ID 12: INPIONCH1 exon 5 amino acid sequence | |
| 1 | LWVDWRDQSP QARVDQDGHV KLNLALATET NCNFELLHFP RDHSNCSLSF YALSNTA |
| SEQ ID 13: INPIONCH1 exon 6 DNA sequence | |
| 1 | CGATGGAGTT AGAGTTCCAG GCCCACGTGG TGAACGAGAT TGTGAGTGTC AAGAGGGAAT |
| 61 | ACGTAGTTTA TGATCTGAAG ACCCAAGTCC CACCCCAGCA GCTGGTGCCC TGCTTCCAGG |
| 121 | TGACG |
| SEQ ID 14: INPIONCH1 exon 6 amino acid sequence | |
| 1 | MELEFQAHVV NEIVSVKREY VVYDLKTQVP PQQLVPCFQV T |
| SEQ ID 15: INPIONCH1 exon 7 DNA sequence | |
| 1 | CTGAGGCTGA AGAACACGGC GCTCAAGTCC ATCATCGCTC TCTTGGTGCC TGCAGAGGCA |
| 61 | CTGCTGTTGG CTGACGTGTG CGGGGGGTTG CTGCCCCTCC GGGCCATTGA GCGCATAGGC |
| 121 | TACAAGGTGA CATTGCTGCT GAGTTACCTC GTCCTCCACT CCTCCCTGGT GCAGGCCCTG |
| 181 | CCCAGCTCCT CCTCCTGCAA CCCACTGCTC A |
| SEQ ID 16: INPIONCH1 exon 7 amino acid sequence | |
| 1 | LRLKNTALKS IIALLVPAEA LLLADVCGGL LPLRAIERIG YKVTLLLSYL VLHSSLVQAL |
| 61 | PSSSSCNPLL I |
| SEQ ID 17: INPIONCH1 exon 8 DNA sequence | |
| 1 | TTTACTACTT CACCATCCTG CTGCTGCTGC TCTTCCTCAG CACCATAGAG ACTGTGCTGC |
| 61 | TGGCTGGGCT GCTGGCCCGG GGCAACCTTG GGGCCAAGAG CGGCCCCAGC CCAGCCCCGA |
| 121 | GAGGGGAACA GCGAGAGCAC GGCAACCCAG GGCCTCATCC TGCTGAAG |
| SEQ ID 18: INPIONCH1 exon 8 amino acid sequence | |
| 1 | YYFTILLLLL FLSTIETVLL AGLLARGNLG AKSGPSPAPR GEQREHGNPG PHPAEE |
| SEQ ID 19: INPIONCH1 exon 9 DNA sequence | |
| 1 | AGCCCTCCAG AGGAGTAAAG GGGTCACAGA GAAGCTGGCC TGAGACTGCT GACCGCATCT |
| 61 | TCTTCCTCGT GTATGTGGTT GGGGTGCTGT GCACCCAATT CGTCTTTGCA GGAATCTGGA |
| 121 | TGTGGGCAGC GTGCAAGTCT GACGCAGCCC CTGGAGAGGC TGCACCCCAT GGCAGGCGGC |
| 181 | CTAGACTGTA A |
| SEQ ID 20: INPIONCH1 exon 9 amino acid sequence | |
| 1 | PSRGVKGSQR SWPETADRIF FLVYVVGVLC TQFVFAGIWM WAACKSDAAP GEAAPHGRRP |
| 61 | RL |
| SEQ ID 21: INPIONCH1-5HT3a chimera | |
| 1 | atggccctat ggtccctgct ccatctcacc ttcctggggt tcagcattac cttgctgttg |
| 61 | gtccacgggc agggcttcca agggacagca gccatctggc catccctctt caacgtcaac |
| 121 | ttgtccaaga aggttcagga aagcatccag atcccgaaca atgggagtgc gcccctgctc |
| 181 | gtggatgtgc gggtgtttgt ctccaacgtg tttaatgtgg acatcctgcg atacacaatg |
| 241 | tcctccatgc tgctgcttag gctgtcctgg ctggacactc gcctggcctg gaacactagt |
| 301 | gcacacccgc ggcacgccat cacgctgccc tgggagtctc tctggacacc aaggctcacc |
| 361 | atcctggagg cgctctgggt ggactggagg gaccagagcc cccaggctcg agtagaccag |
| 421 | gacggccacg tgaagctcaa cctggccctc gccacggaga ccaactgcaa ctttgagctc |
| 481 | ctccacttcc cccgggacca cagcaactgc agcctcagct tctacgctct cagcaacacg |
| 541 | gcgatggagt tagagttcca ggcccacgtg gtgaacgaga ttgtgagtgt caagagggaa |
| 601 | tacgtagttt atgatctgaa gacccaagtc ccaccccagc agctggtgcc ctgcttccag |
| 661 | gtgacgctga ggctgaagaa cacggcggaa ttccggcggc ccctcttcta tgtggtcagc |
| 721 | ctgctactgc ccagcatctt cctcatggtc atggacatcg tgggcttcta cctgcccccc |
| 781 | aacagtggcg agagggtctc tttcaagatt acactcctcc tgggctactc ggtcttcctg |
| 841 | atcatcgttt ctgacacgct gccggccact gccatcggca ctcctctcat tggtgtctac |
| 901 | tttgtggtgt gcatggctct gctggtgata agtttggccg agaccatctt cattgtgcgg |
| 961 | ctggtgcaca agcaagacct gcagcagccc gtgcctgctt ggctgcgtca cctggttctg |
| 1021 | gagagaatcg cctggctact ttgcctgagg gagcagtcaa cttcccagag gcccccagcc |
| 1081 | acctcccaag ccaccaagac tgatgactgc tcagccatgg gaaaccactg cagccacatg |
| 1141 | ggaggacccc aggacttcga gaagagcccg agggacagat gtagccctcc cccaccacct |
| 1201 | cgggaggcct cgctggcggt gtgtgggctg ctgcaggagc tgtcctccat ccggcaattc |
| 1261 | ctggaaaagc gggatgagat ccgagaggtg gcccgagact ggctgcgcgt gggctccgtg |
| 1321 | ctggacaagc tgctattcca catttacctg ctggcggtgc tggcctacag catcaccctg |
| 1381 | gttatgctct ggtccatctg gcagtacgct c |
| SEQ ID 22: 5HT3a-INPIONCH1 chimera | |
| 1 | atgctgctgt gggtccagca ggcgctgctc gccttgctcc tccccacact cctggcacag |
| 61 | ggagaagcca ggaggagccg aaacaccacc aggcccgctc tgctgaggct gtcggattac |
| 121 | cttttgacca actacaggaa gggtgtgcgc cccgtgaggg actggaggaa gccaaccacc |
| 181 | gtatccattg acgtcattgt ctatgccatc ctcaacgtgg atgagaagaa tcaggtgctg |
| 241 | accacctaca tctggtaccg gcagtactgg actgatgagt ttctccagtg gaaccctgag |
| 301 | gactttgaca acatcaccaa gttgtccatc cccacggaca gcatctgggt cccggacatt |
| 361 | ctcatcaatg agttcgtgga tgtggggaag tctccaaata tcccgtacgt gtatattcgg |
| 421 | catcaaggcg aagttcagaa ctacaagccc cttcaggtgg tgactgcctg tagcctcgac |
| 481 | atctacaact tccccttcga tgtccagaac tgctcgctga ccttcaccag ttggctgcac |
| 541 | accatccagg acatcaacat ctctttgtgg cgcttgccag aaaaggtgaa atccgacagg |
| 601 | agtgtcttca tgaaccaggg agagtgggag ttgctggggg tgctgcccta ctttcgggag |
| 661 | ttcagcatgg aaagcagtaa ctactatgca gaaatgaagt tctatgtggt catccgcgaa |
| 721 | ttcctcaagt ccatcatcgc tctcttggtg cctgcagagg cactgctgtt ggctgacgtg |
| 781 | tgcggggggt tgctgcccct ccgggccatt gagcgcatag gctacaaggt gacattgctg |
| 841 | ctgagttacc tcgtcctcca ctcctccctg gtgcaggccc tgcccagctc ctcctcctgc |
| 901 | aacccactgc tcatttacta cttcaccatc ctgctgctgc tgctcttcct cagcaccata |
| 961 | gagactgtgc tgctggctgg gctgctggcc cggggcaacc ttggggccaa gagcggcccc |
| 1021 | agcccagccc cgagagggga acagcgagag cacggcaacc cagggcctca tcctgctgaa |
| 1081 | gagccctcca gaggagtaaa ggggtcacag agaagctggc ctgagactgc tgaccgcatc |
| 1141 | ttcttcctcg tgtatgtggt tggggtgctg tgcacccaat tcgtctttgc aggaatctgg |
| 1201 | atgtgggcag cgtgcaagtc tgacgcagcc cctggagagg ctgcacccca tggcaggcgg |
| 1261 | cctagactg |

SEQ ID 23: INPIONCH1-5HT3a chimera
MALWSLLHLTFLGFSITLLLVHGQGFQGTAAIWPSLFNVNLSKKVQESIQIPNNGSAPLLVDVRVFVSNVFNVDILRYT
MSSMLLLRLSWLDTRLAWNTSAHPRHAITLPWESLWTPRLTILEALWVDWRDQSPQARVDQDGHVKLNLALATETNCNF
ELLHFPRDHSNCSLSFYALSNTAMELEFQAHVVNEIVSVKREYVVYDLKTQVPPQQLVPCFQVTLRLKNTAEFRRPLFY
VVSLLLPSIFLMVMDIVGFYLPPNSGERVSFKITLLLGYSVFLIIVSDTLPATAIGTPLIGVYFVVCMALLVISLAETI
FIVRLVHKQDLQQPVPAWLRHLVLERIAWLLCLREQSTSQRPPATSQATKTDDCSAMGNHCSHMGGPQDFEKSPRDRCS
PPPPPREASLAVCGLLQELSSIRQFLEKRDEIREVARDWLRVGSVLDKLLFHIYLLAVLAYSITLVMLWSIWQYA
SEQ ID NO: 24: 5HT3a-INPIONCH1 chimera
MLLWVQQALLALLLPTLLAQGEARRSRNTTRPALLRLSDYLLTNYRKGVRPVRDWRKPTTVSIDVIVYAILNVDEKNQV
LTTYIWYRQYWTDEFLQWNPEDFDNITKLSIPTDSIWVPDILINEFVDVGKSPNIPYVYIRHQGEVQNYKPLQVVTACS
LDIYNFPFDVQNCSLTFTSWLHTIQDINISLWRLPEKVKSDRSVFMNQGEWELLGVLPYFREFSMESSNYYAEMKFYVV
IREFLKSIIALLVPAEALLLADVCGGLLPLRAIERIGYKVTLLLSYLVLHSSLVQALPSSSSCNPLLIYYFTILLLLLF
LSTIETVLLAGLLARGNLGAKSGPSPAPRGEQREHGNPGPHPAEEPSRGVKGSQRSWPETADRIFFLVYVVGVLCTQFV
FAGIWMWAACKSDAAPGEAAPHGRRPRL

1. A polypeptide selected from the group consisting of the amino acid sequence as recited in SEQ ID NO:2,
a fragment of SEQ ID NO:2 having 5-HT3 protein function,
a fragment having an antigenic determinant in common with SEQ ID NO: 2,
a functional equivalent of the amino acid sequence of SEQ ID NO:2, a functional equivalent of a fragment of SEQ ID NO:2 having 5-HT3 protein function, and functional equivalent of a fragment having an antigenic determinant in common with SEQ ID NO:2.

2. A polypeptide or a functional equivalent which is a 5-HT3 receptor subunit.

3. The polypeptide or the functional equivalent according to claim 2 which forms a homopentamer.

4. The polypeptide or the functional equivalent according to claim 2 which forms a heteropentamer.

5. The polypeptide or the functional equivalent according to claim 4, wherein the heteropentamer includes subunits from other ligand-gated ion channels.

6. The polypeptide or the functional equivalent according to claim 5, wherein the heteropentamer includes subunits from other 5-HT3 receptors.

7. The polypeptide or the functional equivalent according to claim 1 which polypeptide is homologous to the amino acid sequence as recited in SEQ ID NO:2, and has 5-HT3 receptor activity.

8. (canceled)

9. The functional equivalent according to claim 1, which exhibits significant structural homology with a polypeptide having the amino acid sequence given in SEQ ID NO:2.

10. The fragment as recited in claim 1 having an antigenic determinant in common with SEQ ID NO:2, which consists of 7 or more amino acid residues from the sequence SEQ ID NO:2.

11. The fragment according to claim 10 comprising amino acid residues 24 to 421 of SEQ ID NO:2.

12. The fragment according to claim 10 comprising amino acid residues 24 to 229 of SEQ ID NO:2.

13. A fusion protein selected from the group consisting of
a ligand binding domain derived from a polypeptide according to claim 1 and a transmembrane domain derived from another member of the 5-HT3 receptor group
and a transmembrane domain derived from a polypeptide according to claim 1 and a ligand binding domain derived from another member of the 5-HT3 receptor group.

14. The fusion protein according to claim 13, comprising the amino acid sequence as recited in SEQ ID NO:23 or SEQ ID NO:24.

15. A purified nucleic acid molecule which encodes a polypeptide, a fragment, or a functional equivalent according to claim 1.

16. The purified nucleic acid molecule according to claim 15, which has the nucleic acid sequence as recited in SEQ ID NO:1, or is a redundant equivalent or fragment thereof.

17. A purified nucleic acid molecule which hybridises under high stringency conditions with a nucleic acid molecule according to claim 15.

18. A vector comprising a nucleic acid molecule as recited in claim 15.

19. A host cell transformed with a vector according to claim 18.

20. A ligand which binds specifically to, and which modulates the activity of, a polypeptide according to claim 1.

21. A ligand which binds specifically to the ligand binding domain or the pore forming domain of a polypeptide according to claim 1.

22. A ligand according to claim 21, which is an antibody.

23. A compound that either increases or decreases the level of expression or activity of a polypeptide according to claim 1.

24. A compound according to claim 23 that does not induce any of the biological effects of the polypeptide.

25. A compound according to claim 24, which is a natural or modified substrate, ligand, enzyme, receptor or structural functional mimetic.

26. (canceled)

27. A method of detecting a disease in a patient, comprising the steps of assessing the level of expression of, or assessing the activity of, a polypeptide according to claim 1 in tissue from said patient and comparing said level of expression or activity to a control level, wherein a level that is different to said control level is indicative of disease.

28. The method according to claim 27 that is carried out in vitro.

29. The method according to claim 27, which comprises the steps of: (a) contacting a ligand with a biological sample under conditions suitable for the formation of a ligand polypeptide complex; and (b) detecting said complex.

30. The method according to claim 27, further comprising the steps of:
a) contacting a sample of tissue from the patient with a nucleic acid probe under stringent conditions that allow the formation of a hybrid complex between a nucleic acid molecule and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample indicative of disease.

31. A method according to claim 27, comprising:
a) contacting a sample of nucleic acid from tissue of the patient with a nucleic acid primer under stringent conditions that allow the formation of a hybrid complex between a nucleic acid molecule and the primer;
b) contacting a control sample with said primer under the same conditions used in step a); and
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease.

32. The method according to claim 27 comprising:
a) obtaining a tissue sample from a patient being tested for disease;
b) isolating a nucleic acid molecule from said tissue sample; and
c) diagnosing the patient for disease by detecting the presence of a mutation which is associated with disease in the nucleic acid molecule as an indication of the disease.

33. The method of claim 32, further comprising amplifying the nucleic acid molecule to form an amplified product and detecting the presence or absence of a mutation in the amplified product.

34. The method of claim 32, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

35. The method according to claim 27, wherein said disease includes, but is not limited to, nausea, vomiting, pain, eating disorders, alcoholism, psychosis, side effects of various anticancer therapies, irritable bowel syndrome, gastrointestinal related disorders, Alzheimer's disease, Parkinson's disease, Huntingtons Chorea, cognitive disorders, behavioral disorders and phobias such as anxiety related illnesses and addiction, obsessive compulsive behavior, memory and learning disorders, depression and panic disorders, asthma, inflammation, sexual dysfunction, disorders of the neuroendocrine and cardiovasular systems.

36. The method according to claim 27, wherein said disease is a disease associated with T cells.

37. The disease of claim 36, wherein the disease is cancer or HIV infection.

38. A pharmaceutical composition comprising a polypeptide according to claim 1, a nucleic acid molecule according to claim 15, a vector according to claim 18, a host cell according to claim 19, a ligand according to claim 20, or a compound according to claim 23.

39. A vaccine composition comprising a polypeptide according to any claim 1 or a nucleic acid molecule according to claim 15.

40. (canceled)

41. The pharmaceutical composition of claim 38 administered for the treatment of diseases associated with T cells, T cell proliferation, or T cell regulation.

42. A method of treating a disease in a patient, comprising administering to the patient a polypeptide according to any claim 1, a nucleic acid molecule according to claim 15, a vector according to claim 18, a host cell according to claim 19, a ligand according to claim 20, or a compound according to claim 23.

43. The method according to claim 42, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

44. The method according to claim 42, wherein, for diseases in which the expression of the natural gene or activity of the polypeptide is higher in a diseased patient when compared to the level of expression or activity in a healthy patient, the polypeptide, nucleic acid molecule, vector ligand, compound or composition administered to the patient is an agonist.

45. A method of monitoring the therapeutic treatment of disease in a patient, comprising monitoring over a period of time the level of expression or activity of a polypeptide according to claim 1, or the level of expression of a nucleic acid molecule according to claim 15 in tissue from said patient, wherein altering said level of expression or activity over the period of time towards a control level is indicative of regression of said disease.

46. A method for the identification of a compound that is effective in the treatment and/or detection of disease, comprising contacting a polypeptide according to claim 1, or a nucleic acid molecule according to claim 15 with one or more compounds suspected of possessing binding affinity for said polypeptide or nucleic acid molecule, and selecting a compound that binds specifically to said nucleic acid molecule or polypeptide.

47. A kit useful for diagnosing disease comprising a first container containing a nucleic acid probe that hybridises under stringent conditions with a nucleic acid molecule according to claim 15, a second container containing primers useful for amplifying said nucleic acid molecule; and instructions for using the probe and primers for facilitating the diagnosis of disease.

48. The kit of claim 47, further comprising a third container holding an agent for digesting unhybridised RNA.

49. A kit comprising an array of nucleic acid molecules, at least one of which is a nucleic acid molecule according to any claim 15.

50. A kit comprising one or more antibodies that bind to a polypeptide as recited in claim 1 and a reagent useful for the detection of a binding reaction between said antibody and said polypeptide.

51. A transgenic or knockout non-human animal that has been transformed to express higher, lower or absent levels of a polypeptide according to claim 1.

52. A method for screening for a compound effective to treat disease, by contacting a non-human transgenic animal according to claim 51 with a candidate compound and determining the effective of the compound on the disease of the animal.
